(19) United States
(12) Patent Application Publication
(10) Pub. No.: US 2021/0283189 A1
(43) Pub. Date: Sep. 16, 2021

(54) METHODS AND COMPOSITIONS FOR TREATING VIRAL INFECTIONS

(71) Applicant: PLURISTEM LTD., Haifa (IL)

(72) Inventors: Yaacob Yanay, Shimshit (IL); Rachel Ofir, Adi (IL); Marc Tritel, Beit Shemesh (IL)

(73) Assignee: PLURISTEM LTD., Haifa (IL)

(21) Appl. No.: 17/198,308

(22) Filed: Mar. 11, 2021

Related U.S. Application Data
(60) Provisional application No. 62/988,815, filed on Mar. 12, 2020.
(60) Provisional application No. 63/006,117, filed on Apr. 7, 2020.
(60) Provisional application No. 63/049,144, filed on Jul. 8, 2020.

(30) Foreign Application Priority Data
May 14, 2020 (IL) 274697

(51) Int. Cl. A61K 35/50; C07K 14/705; C12N 5/00; C12N 5/073; A61P 31/12

(52) U.S. Cl. CPC A61K 35/50; C07K 14/7055; C07K 14/70596; A61P 31/12; C12N 5/0062; C12N 5/0605; C12N 5/0075

(57) ABSTRACT

Disclosed herein are methods and compositions comprising placental adherent stromal cells for treating viral infections and sequelae thereof.

### FIELD

Disclosed herein are methods and compositions for treating viral infections.

### BACKGROUND

Viruses are obligatory intracellular parasites. Animal viruses must cross the host boundary for cell entry and exit. In enveloped viruses, this occurs by fusion of the incoming virus with, and budding of the nascent virus through a cellular membrane. In nonenveloped viruses, virus entry requires transient disturbance of a cellular (mostly endosomal) membrane to transfer the viral genome into the cytoplasm. Intracellularly, viruses induce cytoplasmic membrane structures and compartments, in which genome replication and assembly occurs.

Viral infections account for large fraction of infectious disease mortality and morbidity worldwide. Cytomegalovirus (CMV), for example, is a beta herpesvirus; it is a major cause of morbidity and mortality in immunocompromised individuals, including AIDS patients and recipients of hematopoietic stem cell transplantation (HSCT) or solid organ transplants, cancer patients, and patients at intensive care. CMV is also the leading cause of congenital infection, affecting about 1% of live births, with resultant neurological damage and loss of hearing. Despite the considerable public health burden of congenital CMV, no established prenatal antiviral treatments are available.

Acquired immunodeficiency syndrome (AIDS) is the result of infection by human immunodeficiency virus (e.g., HIV-1). HIV continues to be a major global public health issue. Current therapy for HIV-infected individuals typically consists of a combination of approved anti-retroviral agents. Over two dozen drugs are currently approved for HIV infection, either as single agents or as fixed dose combinations or single tablet regimens. Despite the armamentarium of agents and drug combinations, there remains a medical need for new anti-retroviral agents, due in part to the need for chronic dosing to combat infection. Significant problems related to long-term toxicities are documented, creating a need to address and prevent these co-morbidities.

HCV is an RNA virus belonging to the Hepacivirus genus in the Flaviviridae family. The HCV virion contains a positive-stranded RNA genome encoding all known virus-specific proteins in a single, uninterrupted, open reading frame. The open reading frame comprises approximately 9500 nucleotides and encodes a single large polyprotein of about 3000 amino acids (AA). The polyprotein comprises a core protein, envelope proteins E1 and E2, a membrane bound protein p7, and the non-structural proteins NS2, NS3, NS4A, NS4B, NS5A and NS5B. Chronic HCV infection is associated with progressive liver pathology, including cirrhosis and hepatocellular carcinoma. Chronic hepatitis C may be treated with peginterferon-alpha in combination with ribavirin, which exhibits substantial limitations to efficacy and tolerability. There is a need for new therapies to treat HCV infection.

HBV infection is a major public health problem, affecting approximately 2 billion people worldwide. Among them, 350 million people worldwide and 1.4 million in the US develop a chronic infection, which can lead to chronic persistent hepatitis, liver cirrhosis, and hepatocellular carcinoma (HCC). Every year hundreds of thousands of people die from end stage liver disease caused by HBV infection. The burden of chronic HBV infection continues to be a significant unmet worldwide medical problem, due to suboptimal treatment options and sustained rates of new infections in most parts of the developing world.

Herpes simplex virus (HSV) is a neurotropic pathogen, and transits to sensory nerves after initial infection into mucosal epithelium, then latently infects for a lifetime at trigeminal ganglion or sacral ganglion. Latent HSV sometimes reactivates, causing a variety of pathologies. Two serotypes (HSV-1, HSV-2) are known for HSV. HSV-1 predominantly causes lip/corneal herpes, and HSV-2 predominantly causes genital herpes. Antiviral therapies against these viruses are sorely lacking.

Dengue virus (DENV) is one of the most significant mosquito-borne viral infections affecting humans today and is an NIAID Category A Biodefense pathogen. DENV is a plus-stranded RNA virus and a member of the Flaviviridae family. The 4 Dengue virus serotypes (DENV1, DENV2, DENV3, and DENV4) are defined by the viral envelope protein (E) and share 60% sequence homology at the AA level.

Filoviruses (e.g., Ebola virus (EBOV) and Marburg virus (MARV)) are among the most lethal and destructive viruses. They cause severe, often fatal viral hemorrhagic fevers in humans and nonhuman primates (e.g., monkeys, gorillas, and chimpanzees). Filoviruses are of particular concern as possible biological weapons since they have the potential for aerosol dissemination and weaponization.

### SUMMARY

Provided herein are methods and compositions for treating viral infections, comprising administration of placental adherent stromal cells (ASC), and conditioned media (CM) thereof.

Conditioned medi[a]/[um]/CM, as used herein, refers to a growth medium that has been used to incubate a cell culture. The present disclosure is not intended to be limited to particular medium formulations; rather, any medium suitable for incubation of placental ASC is encompassed.

In certain embodiments, the described placental ASC have been cultured on a 2-dimensional (2D) substrate, a 3-dimensional (3D) substrate, or a combination thereof. Non-limiting examples of 2D and 3D culture conditions are provided in the Detailed Description and in the Examples.

Alternatively or in addition, the placental ASC are allogeneic to the subject; or, in other embodiments, are autologous; or, in other embodiments, are xenogeneic

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings.

FIG. 1 is a diagram of a bioreactor that can be used to prepare the cells.

FIG. 2 contains pictures of bone marrow (BM)-derived MSC (top row) or placental cells after adipogenesis assays. Cells were incubated with (left column) or without (right column) differentiation medium. Placental ASC were expanded in SRM (middle 3 rows depict 3 different batches) or in full DMEM (bottom row).

FIG. 3 contains pictures of BM-derived MSC (top row) or placental cells after osteogenesis assays. Cells were incubated with (left column) or without (right column) differentiation medium. Placental ASC were expanded in SRM (middle 3 rows depict 3 different batches) or in full DMEM (bottom row).

FIGS. 4A-J are plots of luminescence of Luminex® beads, reflective of concentration (vertical axis), for IL-1-ra, Collagen IV-1a, Fibronectin, IL-13, HGF, VEGF-A, IL-4, PDGF-AA, TIMP-1, TGFb2, and TGFb1 (in A-J, respectively) in conditioned medium batches. P250416 R21 and P150518 R02 are maternal batches; R090418 R01 and R170216 R19 are fetal/serum batches; and PD060918 437B R01 and PD08016 441 BR09 (also labeled as “PD08016 441B BR09”) are fetal SF batches. Bioreactor media from various batches (horizontal axis) were subjected to no treatment (BR, lanes 1-6 from left), Tangential Flow Filtration (TFF; Pall Corporation; lanes 7-12), or lyophilization (LYP; lanes 13-18) (upper panels). Lower panels depict analyses of CM generated in plates, with a higher cell/medium ratio.

FIG. 5 is a graph of secretion of IL-10 by PBMC in the absence or presence of ASC. Bars in each group, from left to right are: 1-3: Rat IL-10 after stimulation with 0, 1, or 10 mcg/ml LPS; and 4-6: human IL-10 after stimulation with 0, 1, or 10 mcg/ml LPS.

FIGS. 6A-B are charts depicting lymphocyte proliferation, measured by [³H]thymidine incorporation. Three replicates of each sample were performed. A. 2×10⁵ peripheral blood (PB)-derived MNC (donor A) were stimulated with an equal number of irradiated (3000 Rad) PB-derived MNCs (donor B) in an MLR test, in the presence of different amounts of ASC. B. PB-derived MNCs stimulated with ConA (1.5 mg/ml).

FIGS. 7A-C are charts depicting ASC regulation of pro- and anti-inflammatory cytokine secretion by human MNCs (isolated from peripheral blood). A-B depict secretion of IFN-gamma (A) and TNF-alpha (B) stimulation with ConA. C depicts secretion of IFN-gamma, TNF-alpha and IL-10 (left, middle, and right bars in each series, respectively) following stimulation with LPS. Supernatants were analyzed by ELISA

FIG. 8 is a graph of secretion profile of ASC under normoxic or hypoxic conditions.

FIGS. 9A-B are graphs (each split into 2 panels) depicting secretion, measured by fluorescence, of various factors following incubation of ASC with TNF-alpha+IFN-gamma (gray bars) or control media (black bars) in two separate experiments. C-D are graphs depicting fold-increase of secretion, measured by fluorescence, of GRO, IL-8, MCP-1, and RANTES (C), and IL-6, MCP-3, Angiogenin, Insulin-like Growth Factor Binding Protein-2 (IGFBP-2), Osteopontin, and Osteoprotegerin (D) following incubation of ASC with TNF-alpha alone, relative to incubation with control media (no cytokines).

FIGS. 10A-B are graphs depicting fold-increase relative to control medium (containing no cytokines) in secretion of MCP-1 (A) and GM-CSF (B) in several experiments, as measured by ELISA.

FIGS. 11A-B are plots of average (A) or individual (B) CRP levels (vertical axis) vs. days from ASC administration (horizontal axis). In B, data from individual subjects are represented by different symbols and/or line patterns. Levels after the first and second (where applicable) administration are shown as black and gray lines, respectively.

FIGS. 12A-B are plots of PEEP (Positive End Expiratory Pressure; A) and pH (B)(vertical axis) vs. days from ASC administration (horizontal axis).

FIGS. 13A-B are chest radiographs of a patient showing improvement after (B) vs. before (A) ASC administration.

FIG. 14 is a plot of creatinine (vertical axis) vs. days from ASC administration (horizontal axis).

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise placental adherent stromal cells (ASC) and their conditioned media (CM). In some embodiments, the ASC may be human ASC, or in other embodiments animal ASC.

In one embodiment, there is provided a method for treating, or in another embodiment reducing an incidence of, or in another embodiment ameliorating, a viral infection, comprising administering a composition that comprises a cultured placental ASC, thereby treating, reducing an incidence of, or ameliorating a viral infection. In another embodiment, there is provided a composition that comprises a cultured placental ASC, for treating, reducing an incidence of, or ameliorating a viral infection.

In various embodiments, the placental ASC are maternal tissue-derived ASC (ASC from a maternal portion of the placenta); fetal tissue-derived ASC (ASC from a fetal portion of the placenta); or a mixture thereof. Alternatively or in addition, the placental ASC are allogeneic to the subject; or, in other embodiments, are autologous; or, in other embodiments, are xenogeneic. In certain embodiments, the composition is an injected composition, e.g., intramuscularly injected.

In one embodiment, there is provided a method for treating, or in another embodiment reducing an incidence of, or in another embodiment ameliorating, a viral infection, comprising administering a composition that comprises a cultured ASC, wherein said cultured ASC have been incubated on a 3D substrate, thereby treating, reducing an incidence of, or ameliorating a viral infection. In another embodiment, there is provided a composition that comprises a 3D-cultured ASC, for treating, reducing an incidence of, or ameliorating a viral infection.

In another embodiment, there is provided a method for preventing, ameliorating, or reducing an incidence of deterioration of a subject with a viral infection, comprising administering a composition that comprises a cultured placental ASC, thereby preventing, ameliorating, or reducing an incidence of deterioration of a subject with a viral infection. In another embodiment, there is provided a composition that comprises a cultured placental ASC, for preventing, ameliorating, or reducing an incidence of deterioration of a subject with a viral infection.

In yet another embodiment, there is provided a method for treating, or in another embodiment reducing an incidence of, or in another embodiment ameliorating, sepsis resulting from a viral infection, comprising administering a composition that comprises a cultured placental ASC, thereby treating, reducing an incidence of, or ameliorating sepsis. In another embodiment, there is provided a composition that comprises a cultured placental ASC, for treating, reducing an incidence of, or ameliorating sepsis resulting from a viral infection. In certain embodiments, the sepsis is secondary to pneumonia, which may in turn be secondary to a viral infection. In specific embodiments, the sepsis is severe sepsis.

In yet another embodiment, there is provided a method for treating, or in another embodiment reducing an incidence of, or in another embodiment ameliorating, sepsis resulting from a viral infection, comprising administering a composition that comprises a cultured ASC, wherein said cultured ASC have been incubated on a 3D substrate, thereby treating, reducing an incidence of, or ameliorating sepsis. In another embodiment, there is provided a composition that comprises a 3D-cultured ASC, for treating, reducing an incidence of, or ameliorating sepsis resulting from a viral infection. In certain embodiments, the sepsis is secondary to pneumonia, which may in turn be secondary to a viral infection. In specific embodiments, the sepsis is severe sepsis.

In another embodiment, there is provided a method for treating, or in another embodiment reducing an incidence of, or in another embodiment ameliorating, multiple organ dysfunction syndrome resulting from a viral infection, comprising administering a composition that comprises a cultured placental ASC, thereby treating, reducing an incidence of, or ameliorating multiple organ dysfunction syndrome. In another embodiment, there is provided a composition that comprises a cultured placental ASC, for treating, reducing an incidence of, or ameliorating multiple organ dysfunction syndrome resulting from a viral infection. In certain embodiments, the multiple organ dysfunction syndrome is secondary to pneumonia, which may in turn be secondary to a viral infection.

In still other embodiments, there is provided a method for treating, or in another embodiment reducing an incidence of, or in another embodiment ameliorating, gastrointestinal injury resulting from a viral infection, comprising administering a composition that comprises a cultured placental ASC, thereby treating, reducing an incidence of, or ameliorating gastrointestinal injury. In another embodiment, there is provided a composition that comprises a cultured placental ASC, for treating, reducing an incidence of, or ameliorating gastrointestinal injury resulting from a viral infection.

In other embodiments, there is a provided a method of treating or reducing an incidence of a complication of a viral infection, comprising administering a composition that comprises a cultured placental ASC. In certain embodiments, the described complication is at least one of vasculitis, lymphadenopathy, or both (a non-limiting example of which is Kawasaki disease). In other embodiments, the complication is pediatric multisystem inflammatory syndrome.

In other embodiments, there is a provided a method of treating or reducing an incidence of a complication of a viral infection, comprising administering a composition that comprises a cultured ASC, wherein said ASC have been incubated on a 3D substrate. In certain embodiments, the described complication is at least one of vasculitis, lymphadenopathy, or both (a non-limiting example of which is Kawasaki disease). In other embodiments, the complication is pediatric multisystem inflammatory syndrome.

In still other embodiments, there is provided a method for treating, preventing, or ameliorating systemic inflammation, comprising administering a composition that comprises a cultured placental ASC, wherein said inflammation is associated with a viral infection, thereby treating, preventing, or ameliorating systemic inflammation.

In still other embodiments, there is provided a method for treating, preventing, or ameliorating an after-effect of a viral infection, comprising administering a composition that comprises a cultured placental ASC, thereby treating, preventing, or ameliorating an after-effect of a viral infection. In another embodiment, there is provided a composition that comprises a cultured placental ASC, for treating, reducing an incidence of, or ameliorating an after-effect of a viral infection. Alternatively or in addition, the after-effect is first observed after the patient tests negative (e.g., by a nasal swab) for an active viral infection. In certain embodiments, the after-effect is fatigue. In other embodiments, the after-effect is anxiety. In yet other embodiments, the after-effect is shortness of breath. In still other embodiments, the after-effect is sustained cough. In other embodiments, the after-effect is limb pain (e.g., arm and/or leg pain), which may be, in various embodiments, a burning sensation, a tingling, or a feeling of unease in the affected limb(s), each of which represents a separate embodiment.

In still other embodiments, there is provided a method for treating, preventing, or ameliorating an after-effect of a viral infection, comprising administering a composition that comprises a cultured ASC, wherein said ASC have been incubated on a 3D substrate, hereby treating, preventing, or ameliorating an after-effect of a viral infection. In another embodiment, there is provided a composition that comprises a 3D-cultured ASC, for treating, reducing an incidence of, or ameliorating an after-effect of a viral infection. Alternatively or in addition, the after-effect is first observed after the patient tests negative (e.g., by a nasal swab) for an active viral infection. In certain embodiments, the after-effect is any after-effect mentioned herein, each of which represents a separate embodiment.

In another embodiment, there is provided use of ASC for the manufacture of a medicament for treating or ameliorating any of the diseases, disorders, and complications mentioned herein, each of which represents a separate embodiment.

In still other embodiments, there is provided an article of manufacture, comprising (a) a packaging material, wherein the packaging material comprises a label for use in any of the diseases, disorders, and complications mentioned herein, each of which represents a separate embodiment; and (b) a pharmaceutical composition comprising ASC.

In certain embodiments, there is provided a method of treating a viral infection in a subject in need thereof, the method comprising the step of administering to the subject a therapeutically effective amount of placental ASC, thereby treating the viral infection in the subject. In other embodiments, there is provided a method of suppressing viral replication in a subject in need thereof, the method comprising the step of administering to the subject a composition comprising ASC. In still other embodiments, there is provided a method of reducing a viral load in a subject in need thereof, the method comprising the step of administering to the subject a composition comprising ASC. As provided herein, administration of ASC is useful in treating viral infections. The described ASC secrete anti-viral factors when exposed to an inflammatory environment, for example as may be encountered in a bioreactor or in the context of a viral infection.

In another embodiment, there is provided use of ASC for the manufacture of a medicament for treating a viral infection. In another embodiment is provided use of ASC for the manufacture of a medicament identified for suppressing viral replication. In another embodiment is provided use of ASC for the manufacture of a medicament identified for reducing viral load. In still other embodiments, there is provided a pharmaceutical composition for treating a viral infection, suppressing viral replication, or reducing viral load, comprising the described ASC.

In still other embodiments, there is provided an article of manufacture, comprising (a) a packaging material, wherein the packaging material comprises a label for use in treating a viral infection and (b) a pharmaceutical composition comprising ASC. In other embodiments, a pharmaceutical agent contained within the packaging material, wherein the pharmaceutical agent is effective for the treatment of a subject suffering from a viral infection; and the packaging material comprises a label which indicates that the pharmaceutical agent can be used for treating a viral infection. In some embodiments, the pharmaceutical composition is frozen. In other embodiments, the label indicates use in suppressing viral replication. In still other embodiments, the label indicates use in reducing viral load. As provided herein, administration of a therapeutically effective amount of ASC is useful in treating viral infections.

In certain embodiments, the described virus is selected from: a retrovirus, for example a lentivirus, for example, visna/maedi virus, caprine arthritis encephalitis virus (CAEV), equine infectious anemia virus (EIAV), bovine immunodeficiency virus (BIV), feline immunodeficiency virus (FIV), or HIV-1; the Flaviviridae family, for example hepatitis C (HCV), West Nile virus, Dengue virus (for example DV1 [Dengue virus 1], DV2, DV3, or DV4), Chikungunya virus, Yellow fever virus, Japanese encephalitis virus, Murray Valley encephalitis virus, Rocio virus, West Nile fever virus, St. Louis encephalitis virus, tick-borne encephalitis virus, Louping ill virus, Powassan virus, Omsk hemorrhagic fever virus, and Kyasanur forest disease virus; Caliciviridae, for example Noroviruses and Sapoviruses; Hepadnaviridae, for example hepatitis B virus; a Herpes virus, for example HSV-1, HSV-2, and varicella-zoster virus; a virus belonging to Adenoviridae, Papovaviridae, Anelloviridae, Circoviridae, Parvoviridae, Reoviridae, Birnaviridae, Arenaviridae, Orthomyxoviridae, Paranxoviridae, or Bunyaviridae; a Rhabdovirus, for example rabies, influenza virus; Respiratory syncytial virus (RSV); or a single-stranded RNA virus. In other embodiments, the virus may be selected from Lassa fever virus, lymphocytic choriomeningitis virus, Ebola virus, Marburg virus, cytomegalovirus, Mumps virus, human T-lymphotrophic virus, type 1, equine infectious anemia virus, vesicular stomatitis virus, Epstein-Barr virus, the or rubella virus. In particular embodiments, the virus is a hemorrhagic fever virus. In more specific embodiments, the virus is yellow fever virus; is Dengue virus; is Marburg virus; is Ebola virus; is Lassa virus; is Crimean-Congo HFV; is Rift Valley virus; is Human Immunodeficiency Virus 1 (HIV-1); is hepatitis C virus (HCV); is hepatitis B virus (HBV); is herpes simplex virus 1 (HSV-1), or is herpes simplex virus 2 (HSV-2). In other embodiments, the virus is selected from yellow fever virus, Dengue virus, Marburg virus, Ebola virus, Lassa virus, Crimean-Congo HFV, and Rift Valley virus. In still other embodiments, the virus is selected from HIV-1, HCV, HBV, HSV-1, HSV-2, Dengue virus, Marburg virus, and Ebola virus.

In other embodiments, there is provided a method of treating a Hemorrhagic Fever (HF), the method comprising the step of administering to the subject a therapeutically effective amount of placental ASC, thereby treating the HF in the subject. As provided herein, administration of a therapeutically effective amount of ASC is useful in treating HF. Methods of diagnosing and tracking the progression of HF are well known in the art, and include, inter alia, measurement of serum cytokine levels and/or expression in PBMC of cytokines, chemokines, death proteins such as TRAIL, and/or fibrin-related genes, as described in Bray M et al., 2001, Rubins K H et al., 2007, and the references cited therein. Classically, HF is caused by viruses classified as filoviruses (for example Ebola and Marburg virus), flaviviruses (for example yellow fever virus and Dengue virus), arenaviruses (for example Lassa virus), and bunyaviruses (for example Crimean-Congo HFV and Rift Valley virus). In some cases, a similar syndrome may be caused by other pathogens, for example influenza virus. As provided herein, administration of ASC is useful in treating HF. The described ASC secrete anti-viral factors when exposed to an inflammatory environment, for example as may be encountered in a bioreactor or in the context of a viral infection. Additionally, the ASC secrete anti-inflammatory factors that can ameliorate hypercytokinemia.

In another embodiment is provided use of the described ASC for the manufacture of a medicament identified for treating a hemorrhagic fever. In still other embodiments, there is provided a pharmaceutical composition for treating a hemorrhagic fever, comprising the described ASC.

In other embodiments, there is provided a method of treating hypercytokinemia (also known, in some embodiments, as “cytokine storm”), the method comprising the step of administering to the subject a therapeutically effective amount of ASC, thereby treating the hypercytokinemia in the subject. Methods of diagnosing and tracking the progression of hypercytokinemia are well known in the art, and include, inter alia, measurement of serum cytokine levels and/or expression in PBMC of cytokines, chemokines, and/or death proteins such as TRAIL, as described in Bray M et al., 2001, Yen J Y et al., 2011, and the references cited therein.

In other embodiments, there is provided a method of treating systemic inflammatory response syndrome (SIRS), the method comprising the step of administering to the subject a therapeutically effective amount of ASC, thereby treating the SIRS in the subject. Methods of diagnosing and tracking the progression of SIRS are well known in the art, and include, inter alia, measurement of serum cytokine levels and/or expression in PBMC of cytokines, chemokines, death proteins such as TRAIL, and/or fibrin-related genes as described in Bray M et al., 2001, Geisbert T W et al., 2003, Yen J Y et al., 2011, and the references cited therein.

In another embodiment is provided use of ASC for the manufacture of a medicament identified for treating hypercytokinemia or SIRS. In still other embodiments, there is provided a pharmaceutical composition for treating hypercytokinemia or SIRS, comprising the described ASC.

In certain embodiments, the herein-described SIRS patient exhibits leukopenia, e.g., an absolute WBC count below 3500 cells per microliter [/μL]); or, in other embodiments, below 3000, 2500, 2000, 1500, or 1000 cells/μL, e.g., for an adult. Alternatively, the subject exhibits leukocytosis, e.g., an absolute WBC count above >10,000 cells/μL; or in other embodiments, above 10,500, 11,000, 12,000, 13,000, 15,000, 18,000, or 20,000 cells/μL, e.g., for an adult.

Alternatively or in addition, the SIRS patient exhibits both leukopenia and lymphopenia (83.2%), e.g., a lymphocyte count below 1000/μL; or, in other embodiments, below 800, 600, 500, 400, 300, 200, or 100 cells/μL, e.g., for an adult; and/or (in different embodiments) thrombocytopenia, e.g., a platelet count below 50,000/μL; or, in other embodiments, below 100,000, 80,000, 60,000, 40,000, 30,000, 20,000, or 20,000/μL, e.g., for an adult.

In still other embodiments, the SIRS patient exhibits elevated levels of C-reactive protein; e.g., greater than 3 milligrams per liter (mg/L); or, in other embodiments, greater than 4, 5, 6, 8, or 10 mg/L. Each embodiment of SIRS symptoms and criteria may be freely combined, except where self-contradictory (e.g., leukopenia and leukocytosis).

In other embodiments, there is provided a method of treating dysregulated coagulation, the method comprising the step of administering to the subject a therapeutically effective amount of ASC, thereby treating the dysregulated coagulation in the subject. Methods of diagnosing and tracking the progression of dysregulated coagulation are well known in the art, and include, inter alia, measurement of intravascular coagulation, e.g. using ROTEM® delta, available from Tem International GmbH, or as described in Monaca E et al., 2014; and expression in PBMC of fibrin-related genes, as described in Bray M et al., 2001, Geisbert T W et al., 2003, Yen J Y et al., 2011, and the references cited therein.

In another embodiment is provided use of ASC for the manufacture of a medicament identified for treating dysregulated coagulation. In still other embodiments, there is provided a pharmaceutical composition for treating dysregulated coagulation, comprising the described ASC.

In other embodiments, there is provided a method of treating septic shock, the method comprising the step of administering to the subject a therapeutically effective amount of ASC, thereby treating septic shock in the subject. Methods of diagnosing and tracking the progression of septic shock are well known in the art, and include, inter alia, measurement of expression levels of tissue factor in primate monocytes and/or macrophages, as described in Bray M et al., 2003, and the references cited therein. In some embodiments, the septic shock is secondary to a hemorrhagic fever virus. In other cases, septic shock may be caused by other pathogens, for example influenza virus. In still other cases, septic shock may be independent of a pathogen, or in other embodiments may be of unknown etiology.

In another embodiment is provided use of ASC for the manufacture of a medicament identified for treating septic shock. In still other embodiments, there is provided a pharmaceutical composition for treating septic shock, comprising the described ASC.

In various embodiments, the described placental ASC are maternal tissue-derived ASC; fetal tissue-derived ASC; or a mixture thereof. Alternatively or in addition, the placental ASC are allogeneic to the subject; or, in other embodiments, are autologous; or, in other embodiments, are xenogeneic. In other embodiments, conditioned medium (CM) of placental ASC is utilized in place of ASC. In other embodiments, the composition is an injected composition, e.g., intramuscularly injected. Any of these embodiments may be freely combined with any of the therapeutic embodiments mentioned herein.

As provided herein, placental ASC and CM exhibit ability to ameliorate inflammation, ischemia/reperfusion injury, muscle trauma, irradiation and hematological disorders; to modulate and support recovery of various organ systems, e.g., by inducing regeneration and modulating undesired inflammation; and in treating pulmonary hypertension, lung fibrosis, acute kidney injury, and gastrointestinal injury.

In still other embodiments, there is provided a composition for treating or ameliorating any of the diseases, disorders, and complications mentioned herein, each of which represents a separate embodiment, comprising cultured placental ASC.

In yet other embodiments, there is provided a population of placenta-derived ASC, that secrete elevated levels of RANTES (C-C motif chemokine 5; UniProt No. P13501). In some embodiments, the RANTES secretion is measured after removing the cells from the bioreactor. In certain embodiments, the RANTES secretion is at least 2-fold, at least 3-fold, at least 4-fold, at least 5-fold, at least 6-fold, or at least 8-fold, at least 10-fold, at least 15-fold, at least 20-fold, at least 30-fold, at least 50-fold, at least 70-fold, at least 100-fold, at least 150-fold, at least 200-fold, at least 300-fold, at least 500-fold, at least 700-fold, or at least 1000-fold as high as cells prepared in the absence of added cytokines. In certain embodiments, as provided herein, RANTES secretion is measured by incubating 5×10⁵ ASC for 24 hours under standard conditions, then replacing the medium with serum medium and incubating for an additional 24 hours. In more specific embodiments, at least 20 picrograms (pg), at least 30 pg, at least 50 pg, at least 70 pg, at least 100 pg, at least 150 pg, at least 200 pg, at least 300 pg, at least 400 pg, at least 500 pg, at least 700 pg, at least 1000 pg, at least 1500 pg, at least 2000 pg, at least 3000 pg, at least 4000 pg, at least 5000 pg, at least 6000 pg, at least 8000 pg, at least 10,000 pg, at least 15,000 pg, or at least 20,000 pg, of RANTES are secreted by the cells under these conditions. In still other embodiments, there is provided a culture, comprising the RANTES-secreting ASC, or in other embodiments, a bioreactor comprising the culture. In some embodiments, the bioreactor further comprises a synthetic three-dimensional substrate. In other embodiments, there is provided a composition, comprising the RANTES-secreting ASC. In certain embodiments, the composition further comprises a pharmacologically acceptable excipient. In further embodiments, the excipient is a cryoprotectant, or is a carrier protein. Alternatively or in addition, the composition is frozen. In other embodiments, there is provided a use of these ASC in any of the therapeutic embodiments described herein, each of which represents a separate embodiment.

Basal Media for Expansion of ASC

Those skilled in the art will appreciate that growth media are utilized to expand the described placental ASC and/or produce the described CM for the compositions and methods described herein. Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153, and mixtures thereof in any proportions. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be FBS, or in other embodiments another animal serum.

Alternatively or in addition, the medium may be supplemented by growth factors, vitamins (e.g. ascorbic acid), cytokines, salts (e.g. B-glycerophosphate), steroids (e.g. dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e. the 2D growth medium and the 3D growth medium, may be independently selected from each of the described embodiments relating to medium composition. In various embodiments, any medium suitable for growth of cells in a standard tissue apparatus and/or a bioreactor may be used.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

ASC and Sources Thereof

In certain embodiments, the described ASC are placenta-derived. Except where indicated otherwise, the terms “placenta”, “placental tissue”, and the like, as used herein, refer to any portion of the placenta. Placenta-derived ASC may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer, non-limiting examples of which are phosphate-buffered saline (PBS) and Hank's buffer. In certain embodiments, the placental tissue from which ASC are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. More specific embodiments of decidua are decidua basalis, decidua capsularis, and decidua parietalis. In a non-limiting embodiment, a mixture of maternal and fetal placental cells can be obtained by mincing whole placenta or in other embodiments a portion thereof, or, in still other embodiments, whole placenta, apart from the amnion, chorion, and/or umbilical cord.

Placental cells may be obtained, in various embodiments, from a full-term or pre-term placenta. In some embodiments, the placental tissue is optionally minced, followed by enzymatic digestion. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g. Falcon, Becton, Dickinson, San Jose, Calif.) with washing medium. In some embodiments, the tissue treatment includes use of a DNAse, a non-limiting example of which is Benzonase from Merck.

Optionally, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth), however, a variety of sources of placental tissue or cells may be contemplated by the skilled person. In other embodiments, the placenta is used within 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to an adherent material (e.g., configured as a surface) to thereby isolate adherent cells. In some embodiments, the donor is 35 years old or younger, while in other embodiments, the donor may be any woman of childbearing age.

Placenta-derived cells can be propagated, in some embodiments, by using a combination of 2D and 3D culturing conditions. Conditions for propagating adherent cells in 2D and 3D culture are further described hereinbelow and in the Examples section which follows.

Those skilled in the art will appreciate in light of the present disclosure that cells may be, in some embodiments, extracted from a placenta, for example using physical and/or enzymatic tissue disruption, followed by marker-based cell sorting, and then may be subjected to the culturing methods described herein.

Treatment of Cells with Pro-Inflammatory Cytokines

In certain embodiments of the described methods and compositions, the composition of the medium is not varied during the course of the culturing process used to expand the placental ASC that are used in the described methods and compositions and/or for producing the described CM. In other words, no attempt is made to intentionally vary the medium composition by adding or removing factors or adding fresh medium with a different composition than the previous medium. Reference to varying the composition of the medium does not include variations in medium composition that automatically occur as a result of prolonged culturing, for example due to the absorption of nutrients and the secretion of metabolites by the cells therein, as will be appreciated by those skilled in the art.

In other embodiments, the method used to expand the steps comprises 2D culturing, followed by 3D culturing. In certain embodiments, the 3D culturing method comprises the sub-steps of: (a) incubating ASC in a 3D culture apparatus in a first growth medium, wherein no inflammatory cytokines have been added to the first growth medium; and (b) subsequently incubating the ASC in a 3D culture apparatus in a second growth medium, wherein one or more pro-inflammatory cytokines have been added to the second growth medium. Those skilled in the art will appreciate, in light of the present disclosure, that the same 3D culture apparatus may be used for the incubations in the first and second growth medium by simply adding cytokines to the medium in the culture apparatus, or, in other embodiments, by removing the medium from the culture apparatus and replacing it with medium that contains cytokines. In other embodiments, a different 3D culture apparatus may be used for the incubation in the presence of cytokines, for example by moving (e.g. passaging) the cells to a different incubator, before adding the cytokine-containing medium.

Other embodiments of pro-inflammatory cytokines, and methods comprising same, are described in WO 2017/141181 to Pluristem Ltd, by Zami Aberman et al., which is incorporated by reference herein.

Serum-Free and Serum Replacement Media

In other embodiments, the described cell populations are produced by expanding a population of placental ASC in a medium that contains less than 5% animal serum. In certain embodiments, the cell population contains at least predominantly fetal cells (referred to as a “fetal cell population”), or, in other embodiments, contains at least predominantly maternal cells (a “maternal cell population”). In other embodiments, factors obtained from the maternal, or in other embodiments fetal, cells are used in the described methods and compositions.

In certain embodiments, the aforementioned medium contains less than 4%; less than 3%; less than 2%; less than 1%; less than 0.5%; less than 0.3%; less than 0.2%; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from a variety of species, provided that the serum stimulates expansion of the ASC population. In certain embodiments, the serum is mammalian serum, non-limiting examples of which are human serum, bovine serum (e.g. fetal bovine serum and calf bovine serum), equine serum, goat serum, and porcine serum.

In other embodiments, the described cell populations are produced by a process comprising: a. incubating the ASC population in a first medium, wherein the first medium contains less than 5% animal serum, thereby obtaining a first expanded cell population; and b. incubating the first expanded cell population in a second medium, wherein the second medium also contains less than 5% animal serum, and wherein one or more activating components are added to the second medium. This second medium can also be referred to herein as an activating medium. In other embodiments, the first medium or the second medium, or in other embodiments both the first and second medium, is/are serum free. In still other embodiments, the first medium contains a first basal medium, with the addition of one or more growth factors, collective referred to as the “first expansion medium” (to which a small concentration of animal serum is optionally added); and the activating medium contains a second basal medium with the addition of one or more growth factors (the “second expansion medium”), to which activating component(s) are added. In more specific embodiments, the second expansion medium is identical to the first expansion medium; while in other embodiments, the second expansion medium differs from the first expansion medium in one or more components.

In certain embodiments, the aforementioned step of incubating the ASC population in a first medium is performed for at least 17 doublings, or in other embodiments at least 6, 8, 12, 15, or at least 18 doublings; or 12-30, 12-25, 15-30, 15-25, 16-25, 17-25, or 18-25 doublings.

In other embodiments, the ASC population is incubated in the aforementioned first medium for a defined number of passages, for example 2-3, or in other embodiments 1-4, 1-3, 1-2, or 2-4; or a defined number of population doublings, for example 4-7, or in other embodiments at least 4, at least 5, at least 6, at least 7, at least 8, 4-10, 4-9, 4-8, 5-10, 5-9, or 5-8. The cells are then cryopreserved, then subjected to additional culturing in the first medium. In some embodiments, the additional culturing in the first medium is performed for 6-10 population doublings, or in other embodiments at least 6, at least 7, at least 8, at least 9, at least 10, 6-20, 7-20, 8-20, 9-20, 10-20, 6-15, 7-15, 8-15, 9-15, or 10-15 population doublings. Alternatively, the additional culturing in the first medium is performed for 2-3 passages, or in other embodiments at least 1, at least 2, at least 3, 1-5, 1-4, 1-3, 2-5, or 2-4 passages.

In still other embodiments, the step of incubating the first expanded cell population in a second medium is performed for a defined number of total passages, for example 3-5 passages, or in other embodiments 1-4, 1-3, 2-3, 2-5, or 2-4; or a defined number of total population doublings, for example 12-20, or in other embodiments 12-15, or in other embodiments 15-20, 12-18, 12-16, 14-20, or 14-18 doublings.

In other embodiments, the ASC population is incubated in the second medium for a defined number of days, for example 4-10, 5-10, 6-10, 4-9, 4-8, 4-7, 5-9, 5-8, 5-7, 6-10, 6-9, or 6-8; or a defined number of population doublings, for example at least 3, at least 4, at least 5, at least 6, 3-10, 3-9, 3-8, 4-10, 4-9, or 4-8. The cells are then subjected to additional culturing in the second medium in a bioreactor. In some embodiments, the bioreactor culturing is performed for at least 4, at least 5, at least 6, at least 7, at least 8, at least 9, 4-10, 4-9, 4-8, 5-10, 5-9, 5-8, 6-10, 6-9, or 6-8 population doublings; or, in other embodiments, for at least 4, at least 5, at least 6, at least 7, 4-15, 4-12, 4-10, 4-9, 4-8, 4-7, 4-15, 5-12, 5-10, 5-9, 5-8, 5-7, 6-15, 6-12, 6-10, 6-9, 6-8, or 6-7 days. In certain embodiments, the bioreactor contains 3D carriers, on which the cells are cultured.

In certain embodiments, the aforementioned two-stage incubation is preceded by culturing in a medium containing over 5% animal serum (e.g. as described herein). In general, for such embodiments, the nomenclature of the aforementioned steps is retained. Thus, the first medium (containing less than 5% animal serum) still retains its designation as the “first medium”, and the activating medium retains its designation as the “second [or activating] medium”.

In certain embodiments, the described serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al.

SRM formulations include MSC Nutristem® XF full medium (including the supplement) and MSC Nutristem® XF basal medium (Biological Industries); Stempro® SFM and Stempro® SFM-XF (Thermo Fisher Scientific); PPRF-msc6; D-hESF10; TheraPEAK™ MSCGM-CD™ (Lonza, cat. no. 190632); and MesenCult-XF (Stem Cell Technologies, cat. no. 5429). The StemPro® media contain PDGF-BB, bFGF, and TGF-β, and insulin (Chase et al.). The composition of PPRF-msc6 is described in US 2010/0015710, which is incorporated herein by reference. D-hESF10 contains insulin (10 mcg/ml); transferrin (5 mcg/ml); oleic acid conjugated with bovine albumin (9.4 mcg/ml); FGF-2 (10 ng/ml); and TGF-β1 (5 ng/ml), as well as heparin (1 mg/ml) and standard medium components (Mimura et al.).

In still other embodiments, a chemically-defined medium is utilized. A non-limiting example of a chemically-defined medium contains DMEM/F-12 supplemented with 50 ng/ml PDGF-BB, 15 ng/ml bFGF, and 2 ng/ml TGF-β. This medium yielded similar results to Stempro® SFM-XF. DMEM/F-12 is a known basal medium, available commercially from Thermo Fisher Scientific (cat. no. 10565018).

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

In other embodiments, the described SRM comprises bFGF, TGF-β, and insulin. In additional embodiments, a component selected from transferrin (5 micrograms/milliliter [mcg/ml]) and oleic acid are present; or both transferrin and oleic acid are present. Oleic acid can be, in some embodiments, conjugated with a protein, a non-limiting example of which is albumin. In some embodiments, the SRM comprises 5-20 ng/ml bFGF, 2-10 ng/ml TGF-β, and 5-20 ng/ml insulin, or, in other embodiments, 7-15 ng/ml bFGF, 3-8 ng/ml TGF-β, and 7-15 ng/ml insulin. In more specific embodiments, a component selected from 2-10 mcg/ml transferrin and 5-20 mcg/ml oleic acid, or in other embodiments, a component selected from 3-8 mcg/ml transferrin and 6-15 mcg/ml oleic acid, or in other embodiments the aforementioned amounts of both components (transferrin and oleic acid) is/are also present.

In still other embodiments, the SRM further comprises a component, or in other embodiments 2, 3, or 4 components, selected from ethanolamine, glutathione, ascorbic acid, and albumin. Alternatively or in addition, the SRM further comprises a trace element, or in other embodiments, 2, 3, 4, or more than 4 trace elements. In some embodiments, the trace element(s) are selected from selenite, vanadium, copper, and manganese.

In yet other embodiments, the described SRM comprises bFGF and EGF. In more specific embodiments, the bFGF and EGF are present at concentrations independently selected from 5-40, 5-30, 5-25, 6-40, 6-30, 6-25, 7-40, 7-30, 7-25, 7-20, 8-, 8-17, 8-15, 8-13, 9-20, 9-17, 9-15, 10-15, 5-20, 5-10, 7-13, 8-12, 9-11, or 10 ng/ml. In certain embodiments, insulin: and/or transferrin is also present. In more specific embodiments, the insulin and transferrin are present at respective concentrations of 5-20 and 2-10; 6-18 and 3-8; or 8-15 and 4-7 mcg/ml. Alternatively or in addition, the SRM further comprises an additional component selected from BSA, selenite (e.g. sodium selenite), pyruvate (e.g. sodium pyruvate); heparin, and linolenic acid. In other embodiments 2 or more, or in other embodiments 3 or more, in other embodiments 4 or more, or in other embodiments all 5 of BSA, selenite, pyruvate, heparin, and linolenic acid are present. In more specific embodiments, the BSA, selenite, pyruvate, heparin, and linolenic acid are present at respective concentrations of 0.1-5%, 2-30 ng/mL, 5-25 mcg/ml, 0.05-0.2 mg/ml, and 5-20 nM; or in other embodiments at respective concentrations of 0.2-2%, 4-10 ng/mL, 7-17 mcg/ml, 0.07-0.15 mg/ml, and 7-15 nM; or in other embodiments the aforementioned amounts or 2 or more, or in other embodiments 3 or more, in other embodiments 4 or more, or in other embodiments all 5 of BSA, selenite, pyruvate, heparin, and linolenic acid are present.

In other embodiments, bFGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 8-13, 8-12, 9-11, 9-12, about 10, or 10 nanograms per milliliter (ng/ml).

In other embodiments, EGF, where present, is present at a concentration of 1-40, 1-30, 1-20, 2-40, 2-30, 2-20, 3-40, 3-30, 3-20, 3-15, 4-30, 4-20, 4-15, 5-30, 5-20, 5-15, 6-14, 7-14, 7-25, 7-22, 8-25, 8-22, 9-21, 10-20, 8-13, 8-12, 9-11, 9-12, about 10, or 10 ng/ml.

In other embodiments, TGF-β, where present, is present at a concentration of 1-25, 2-25, 3-25, 4-25, 5-25, 1-20, 1-15, 1-10, 1-8, 1-7, 1-6, 1-5, 2-20, 2-15, 2-10, 3-20, 3-15, 3-10, 3-8, 3-7, 4-8, 4-7, 4-6, 4.5-5.5, about 5, or 5 ng/ml.

In other embodiments, PDGF, where present, is present at a concentration of 1-50, 1-40, 1-30, 1-20, 1-15, 1-10, 1-8, 1-7, 1-6, 1-5, 1-4, 1-3, 1-2, 2-50, 2-40, 2-30, 2-20, 2-15, 2-10, 2-8, 2-7, 2-6, 2-5, 2-4, 3-50, 3-40, 3-30, 3-20, 3-15, 3-10, 3-8, 3-7, 3-6, 3-5, 3-4, 4-40, 4-30, 4-20, 5-40, 5-30, 5-20, 5-15, 5-12, 5-10, 10-20, 10-18, 10-16, or 10-15, 2-20, about 2, about 3, about 5, about 10, about 15, about 20, 2, 3, 5, 10, 15, or 20 ng/mL.

In still other embodiments, ASC are extracted from placenta into serum-containing medium. A non-limiting extraction protocol is described in Example 1 of International Patent Application WO 2016/098061, in the name of Esther Lukasiewicz Hagai el al., published on Jun. 23, 2016, which is incorporated herein by reference in its entirety. Following initial extractions, cells are, in further embodiments, expanded in SRM, in some embodiments for about 2-3 passages, or typically about 4-12 population doublings after the first passage. In yet further embodiments, the culturing is optionally followed by cell concentration, formulation, and cryopreservation, and the optional thawing and additional culturing. In certain embodiments, the initial culturing is all carried out on a 2D substrate. Those skilled in the art will appreciate that non-limiting examples of cryopreservation excipients include DMSO and serum. Other embodiments of cryopreservation media are described herein.

In certain embodiments, the aforementioned culturing steps are followed by culturing in a bioreactor, which is, in some embodiments, performed in SRM. In other embodiments, the bioreactor contains serum-containing medium. In more particular embodiments, the bioreactor culture is performed for 2-5 additional doublings, or in other embodiments up to 10 additional doublings. In certain embodiments, the bioreactor contains a 3D substrate. In other embodiments, a platelet lysate, a non-limiting example of which is human platelet lysate, is used in place of serum. In still other embodiments, a cytokine-containing medium is used in place of the serum-containing medium.

Optionally, bioreactor growth may be followed by any or all of harvest, cell concentration, washing, formulation, and/or cryopreservation.

In other embodiments, the step of incubating the ASC population in a SFM/SPM is performed in a batch culture, and at least a portion of the subsequent step is performed under perfusion. In still other embodiments, the aforementioned subsequent step is initiated in a batch culture for a duration of 2-6, or in other embodiments at least 2, at least 3, at least 4, at least 5, at least 6, 1-5, 2-5, 3-5, 1-2, 1-3, or 1-5-cell doublings, before performing additional expansion in a serum-containing medium under perfusion.

Other SFM and SRM embodiments are disclosed in international patent application publ. no. WO 2019/186471, filed on Mar. 28, 2019, in the name of Lior Raviv et al., which is incorporated herein by reference.

Other Embodiments of Placenta-Derived ASC

In certain embodiments, the described ASC population is plastic adherent under standard culture conditions, express the surface molecules CD105, CD73 and CD90, and do not express CD45, CD34, CD14 or CD11b, CD79α, CD19 and HLA-DR. As used herein, the phrase plastic adherent refers to cells that are capable of attaching to a plastic attachment substrate and expanding or proliferating on the substrate. In some embodiments, the cells are anchorage dependent, i.e., require attachment to a surface in order to proliferate grow in vitro.

In still other embodiments, the described placenta-derived ASC (which hereinafter refers to the cells used in the described methods and compositions, or, in other embodiments, cells used to produce CM, that are used in the described methods and compositions) are a mixture of fetal-derived placental ASC (also referred to herein as “fetal ASC” or “fetal cells”) and maternal-derived placental ASC (also referred to herein as “maternal ASC” or “maternal cells”) and contains predominantly maternal cells. In more specific embodiments, the mixture contains at least 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% maternal cells; or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, or 98-99.5% maternal cells.

In yet other embodiments, the described cells are predominantly or completely maternal cell preparations, or are predominantly or completely fetal cell preparations, each of which represents a separate embodiment. Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g. a Y chromosome in the case of a male fetus), and expanding the cells. In certain embodiments, maternal cell populations are used in the described methods and compositions. In other embodiments, fetal cells are used.

In other embodiments, the described cells are a population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the preparation is a mixture of fetal and maternal cells and is enriched for fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

In more specific embodiments, the mixture contains 20-80%, 30-80%, 40-80%, 50-80%, 60-80%, 20-90%, 30-90%, 40-90%, 50-90%, or 60-90% fetal cells; or 20-80%, 30-80%, 40-80%, 50-80%, 60-80%, 20-90%, 30-90%, 40-90%, 50-90%, or 60-90% maternal cells.

In certain embodiments, the described ASC are distinguishable from human mesenchymal stromal cells (MSC)—which may, e.g., be isolated from bone marrow—as defined by The Mesenchymal and Tissue Stem Cell Committee of the ISCT (Dominici et al., 2006), based on the following 3 criteria: 1. Plastic-adherence when maintained in standard culture conditions (a minimal essential medium+20% fetal bovine serum (FBS)). 2. Expression of the surface molecules CD105, CD73 and CD90, and lack of expression of CD45, CD34, CD14 or CD11b, CD79a or CD19 and HLA-DR. 3. Ability to differentiate into osteoblasts, adipocytes and chondroblasts in vitro. By contrast, the described placental cells are, in certain embodiments, characterized by a reduced differentiation potential, as exemplified and described further herein.

Surface Markers and Additional Characteristics of ASC

Alternatively or additionally, the described ASC (which are used in the described methods and compositions, or to produce CM) may express a marker or a collection of markers (e.g. surface marker) characteristic of MSC or mesenchymal-like stromal cells. In some embodiments, the ASC express some or all of the following markers: CD105 (UniProtKB Accession No. P17813), CD29 (Accession No. P05556), CD44 (Accession No. P16070), CD73 (Accession No. P21589), and CD90 (Accession No. P04216). In some embodiments, the ASC do not express some or all of the following markers: CD3 (e.g., Accession Nos. P09693 [gamma chain] P04234 [delta chain], P07766 [epsilon chain], and P20963 [zeta chain]), CD4 (Accession No. P01730), CD11b (Accession No. P11215), CD14 (Accession No. P08571), CD19 (Accession No. P15391), and/or CD34 (Accession No. P28906). In more specific embodiments, the ASC also lack expression of CD5 (Accession No. P06127), CD20 (Accession No. P11836), CD45 (Accession No. P08575), CD79-alpha (Accession No. B5QTD1), CD80 (Accession No. P33681), and/or HLA-DR (e.g. Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the ASC possess a marker phenotype that is distinct from bone marrow-mesenchymal stem cells (BM-MSC). In certain embodiments, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g. at least 20%) of a population of cells express a marker of interest, and a significant percentage do not express the marker.

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

A cell is said to express a protein or factor if the presence of protein or factor is detectable by standard methods, an example of which is a detectable signal using fluorescence-activated cell sorting (FACS), relative to an isotype control. Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% FBS+2 mM L-Glutamine), added to each well of a 6 well-plate, and cultured for 24 hrs in a humidified incubator (5% CO₂, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

According to some embodiments, the described ASC are capable of suppressing an immune reaction in the subject. Methods of determining the immunosuppressive capability of a cell population are well known to those skilled in the art, and exemplary methods are described in Example 3 of PCT Publication No. WO 2009/144720, which is incorporated herein by reference in its entirety. For example, a mixed lymphocyte reaction (MLR) may be performed. In an exemplary, non-limiting MLR assay, irradiated cord blood (iCB) cells, for example human cells or cells from another species, are incubated with peripheral blood-derived monocytes (PBMC; for example human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively, a similar assay can be performed with peripheral blood (PB)-derived MNC, in place of CB cells. Alternatively or in addition, secretion of pro-inflammatory and anti-inflammatory cytokines by blood cell populations (such as CB cells or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, when 150,000 ASC are co-incubated for 48 hours with 50,000 allogeneic PBMC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC.

In still other embodiments, the ASC secrete immunoregulatory factor(s). In certain embodiments, the ASC secrete a factor selected from TNF-beta (UniProt identifier P01374) and Leukemia inhibitory factor (LIF; UniProt identifier P15018). In other embodiments, the ASC secrete a factor selected from MCP-1 (CCL2), Osteoprotegerin, MIF (Macrophage migration inhibitory factor; Uniprot Accession No. P14174), GDF-15, SDF-1 alpha, GROa (Growth-regulated alpha protein; Uniprot Accession No. P09341), beta2-Microglobulin, IL-6, IL-8 (UniProt identifier P10145), TNF-beta, ENA78/CXCL5, eotaxin/CCL11 (Uniprot Accession No. P51671), and MCP-3 (CCL7). In certain embodiments, the ASC secrete MCP-1, Osteoprotegerin, MIF, GDF-15, SDF-1 alpha, GROa, beta2-Microglobulin, IL-6, IL-8, TNF-beta, and MCP-3, which were found to be secreted by maternal cells. In other embodiments, the ASC secrete MCP-1, Osteoprotegerin, MIF, GDF-15, SDF-1 alpha, beta2-Microglobulin, IL-6, IL-8, ENA78, eotaxin, and MCP-3, which were found to be secreted by fetal cells. All Swissprot and UniProt entries in this paragraph were accessed on Mar. 23, 2017.

In yet other embodiments, the ASC secrete anti-fibrotic factor(s). In certain embodiments, the ASC secrete a factor selected from Serpin E1 (Plasminogen activator inhibitor 1; Uniprot Accession No. P05121) and uPAR (Urokinase plasminogen activator surface receptor; Uniprot Accession No. Q03405). In other embodiments, the ASC secrete factors that facilitate. In still other embodiments, the ASC secrete Serpin E1 and uPAR, which were found to be secreted by maternal and fetal cells. All UniProt entries in this paragraph were accessed on Apr. 3, 2017.

In other embodiments, the ASC secrete a factor(s) that promotes extracellular matrix (ECM) remodeling. In certain embodiments, the ASC secrete a factor selected from TIMP1, TIMP2, MMP-1, MMP-2, and MMP-10. In other embodiments, the ASC secrete TIMP1, TIMP2, MMP-1, MMP-2, and MMP-10, which were found to be secreted by maternal cells. In still other embodiments, the ASC secrete TIMP1, TIMP2, MMP-1, and MMP-10, which were found to be secreted by fetal cells.

In general, in certain embodiments, the described ASC exhibit a spindle shape when cultured under 2D conditions, or more specifically, spindle in shape with a flat, polygonal morphology, and 15-19 μM in diameter. Alternatively or in addition, at least 90% of the cells are Oct-4 minus, as assessed by FACS. In certain embodiments, further steps of purification or enrichment for ASC may be performed. Such methods include, but are not limited to, FACS using ASC marker expression. In other embodiments, the described cells have not been subject to any type of cell sorting in the process used to isolate them. Cell sorting, in this context, refers to any a procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

Alternatively or in addition, the ASC (a) have a Population Doubling Level (PDL) of no more than 25; (b) stimulate endothelial cell proliferation and/or bone marrow migration in in vitro assays (for example, as described herein); (c) secrete, in various embodiments, at least 1, at least 2, at least 3, at least 4, at least 5, at least 6, or all 7 of IL-10, VEGF, Angiogenin, Osteopontin, IL-6, IL-8, MCP-1; (d) exhibit normal karyotype; (e) exhibit expression (in various embodiments, in at least 80%, 85%, 90%, 93%, 95%, 97%, or 98% of the cells) of CD105, CD73, CD29, and CD90; (f) exhibit lack of expression (in various embodiments, in at least 90%, 93%, 95%, 97%, 98%, 99%, 99.5%, 99.7%, or 99.75% of the cells) of CD14, CD19, CD31, CD34, CD45, HLA-DR, and CD235; or any combination of 2 or more of characteristics a-f, each of which represents a separate embodiment. Alternatively or in addition, less than 5%, 10%, 20%, 30%, 40%, or 50%; or more than 60%, 70%, 80%, 90%, or 95% of the cells express CD200. These possibilities may be independently combined with characteristics a-f and combinations thereof, each of which represents a separate embodiment.

In other embodiments, each of CD29, CD73, CD90, and CD105 is expressed by more than 80% of the ASC in each of the populations; and over 90% (or in other embodiments, over 95%, or over 98%) of the cells in each population do not differentiate into osteocytes, after incubation for 17 days with a solution containing 0.1 mcM dexamethasone, 0.2 mM ascorbic acid, and 10 mM glycerol-2-phosphate, in plates coated with vitronectin and collagen (“standard osteogenesis induction conditions”). In yet other embodiments, each of CD34, CD39, and CD106 is expressed by less than 10% of the cells; less than 20% of the cells highly express CD56; and the cells do not differentiate into osteocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the standard conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (“modified osteogenesis induction conditions”). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates, as provided herein. Placental cells expanded as described herein are resistant to osteogenesis, as described in WO 2016/098061, which is incorporated herein by reference

In other embodiments, each of CD29, CD73, CD90, and CD105 is expressed by more than 80% of each of the ASC populations; and over 90% (or in other embodiments, over 95%, or over 98%) of the cells in each population are resistant to adipogenesis, as described in WO 2016/098061, which is incorporated herein by reference. In some embodiments, differentiation into adipocytes is assessed by incubation in adipogenesis induction medium, i.e., a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days (standard adipogenesis induction conditions). In yet other embodiments, each of CD34, CD39, and CD106 is expressed by less than 10% of the cells; less than 20% of the cells highly express CD56; and the cells do not differentiate into adipocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, under the standard conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (modified adipogenic conditions). In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the modified conditions. The aforementioned solutions will typically contain cell culture medium such as DMEM+10% serum or the like, as will be appreciated by those skilled in the art. In various embodiments, the cell population may be less than 50%, less than 40%, less than 30%, less than 20%, or less than 10%, or less than 5% positive for CD200. In other embodiments, the cell population is more than 50%, 60%, 70%, 80%, 90%, 95%, 97%, 98%, 99%, or 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates.

In still other embodiments, the described ASC possess any other marker phenotype, other characteristic (e.g. secretion of factor(s), differentiation capability, resistance to differentiation, inhibition of T-cell proliferation, or stimulation of myoblast proliferation), or combination thereof that is mentioned and/or described in international patent application publ. no. WO 2019/239295, filed Jun. 10, 2019, to Zami Aberman et al, which is incorporated herein by reference.

In still other embodiments, the cells may be allogeneic, or in other embodiments, the cells may be autologous. In other embodiments, the cells may be fresh or, in other embodiments, frozen (for example, cryo-preserved).

In certain embodiments, any of the aforementioned ASC populations are used in the described methods and compositions. In other embodiments, CM obtained from the cells are used in the described methods and compositions. Each population may be freely combined with each of the described treatments, and each combination represents a separate embodiment. Furthermore, the cells utilized to generate CM or contained in the composition can be, in various embodiments, autologous, allogeneic, or xenogenic to the treated subject. Each type of cell may be freely combined with the therapeutic embodiments mentioned herein.

Additional Method Characteristics for Preparation of ASC and CM Derived Therefrom

In some embodiments, the described placental ASC have been incubated in a 3D bioreactor. Each described embodiment for cell expansion may be combined with any of the described embodiments for therapeutic uses of ASC and CM derived therefrom.

In some embodiments, the described ASC or CM are/is harvested from a 3D bioreactor in which the ASC have been incubated. Alternatively or in addition, the cells are cryopreserved, and then are thawed, after which the cells are further expanded and/or CM are isolated therefrom. In other embodiments, after thawing, the cells are cultured in 2D culture, from which the ASC are isolated.

In certain embodiments, the described ASC are, or have been, subject to a 3D incubation, as described further herein. In more specific embodiments, the ASC have been incubated in a 2D adherent-cell culture apparatus, prior to the step of 3D culturing. In some embodiments, ASC are then subjected to prior step of incubation in a 2D adherent-cell culture apparatus, followed by the described 3D culturing steps.

The terms “two-dimensional culture” and “2D culture” refer to a culture in which the cells are exposed to conditions that are compatible with cell growth and allow the cells to grow in a monolayer. An apparatus suitable for such growth is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g. the bioreactor. Certain, non-limiting embodiments of 3D culturing conditions suitable for expansion of adherent stromal cells are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix.

In still other embodiments, the described ASC are, or have been, subject to culturing conditions (e.g. a growth substate, incubation time, bioreactor, seeding density, or harvest density) mentioned in international patent application publ. no. WO 2019/239295, filed Jun. 10, 2019, to Zami Aberman el al, which is incorporated herein by reference.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14 doublings, in other embodiments 5-13 doublings, in other embodiments 5-12 doublings, in other embodiments 5-11 doublings, in other embodiments 5-10 doublings, in other embodiments 6-15 cell doublings, in other embodiments 6-14 doublings, in other embodiments 6-13 doublings, or in other embodiments 6-12 doublings, in other embodiments 6-11 doublings, or in other embodiments 6-10 doublings.

In certain embodiments, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable.

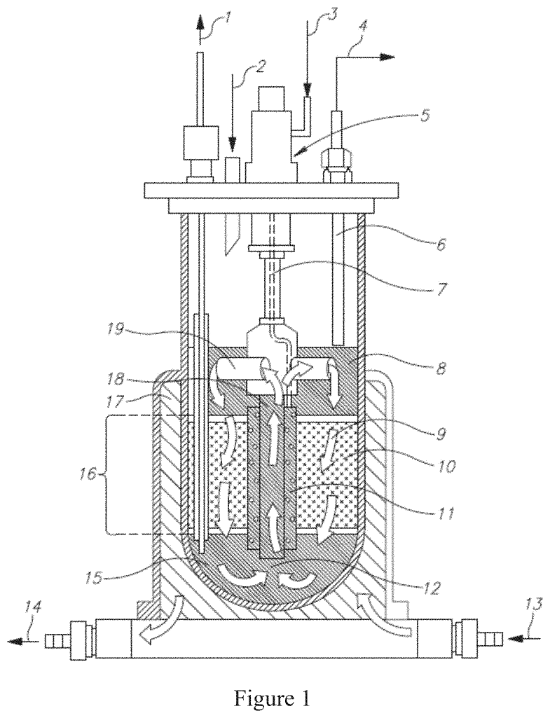

An exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stirring initial rate is used to promote cell attachment, then agitation is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In still other embodiments, the matrix is similar to the Celligen™ Plug Flow bioreactor which is, in certain embodiments, packed with Fibra-Cel® carriers (or, in other embodiments, other carriers).

In other embodiments, prefabricated or rigid scaffolds are utilized. Such scaffolds require, in some embodiments, migration of cells into the scaffold, after cell seeding. In other embodiments, physically crosslinked scaffolds may be utilized, which are, in further embodiments, gels that are formed via reversible changes in pH or temperature.

In other embodiments, microencapsulation is utilized. In certain embodiments, cells are immobilized within a semi-permeable material, e.g., a membrane that allows the diffusion of nutrients, oxygen, and growth factors essential for cell growth.

In certain embodiments, further steps of purification or enrichment for ASC may be performed. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In more particular embodiments, cells may be removed from a 3D matrix while the matrix remains within the bioreactor. In certain embodiments, at least about 10%, 20%, or 30% of the cells are in the S and G2/M phases (collectively), at the time of harvest from the bioreactor.

In certain embodiments, the harvesting process comprises vibration or agitation, for example as described in PCT International Application Publ. No. WO 2012/140519, which is incorporated herein by reference. In certain embodiments, during harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator.

Those skilled in the art will appreciate that a variety of isotonic buffers may be used for washing cells and similar uses. Hank's Balanced Salt Solution (HBSS; Life Technologies) is only one of many buffers that may be used.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Effective dosages and schedules for administering the compositions may be determined empirically, and making such determinations is within the skill in the art. The dosage can be adjusted by the individual physician in the event of any counterindications. A typical dosage of the ASC ranges, in some embodiments, from ^{˜}10 million to ^{˜}500 million cells per administration, depending on the factors mentioned above. For example, the dosage of ASC can be 10, 20, 30, 40, 50, 60, 70, 80, 90, 100, 125, 150, 175, 200, 225, 250, 275, 300, 325, 350, 375, 400, 425, 450, 475, or 500 million cells or any amount in between. It is further understood that a range of ASC can be used including from ^{˜}100 to ^{˜}400 million cells, from ^{˜}150 to ^{˜}300 million cells. Accordingly, disclosed herein are methods of treating, inhibiting, or preventing a viral infection in a subject in need thereof, the method comprising administering to said subject a therapeutically or prophylactically effective amount of ASC, wherein the dosage of ASC administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or from 150 million-300 million cells. ASC, compositions comprising ASC, and/or medicaments manufactured using said ASC can be administered in a single dose, 2 doses, 3 doses, 2-5 doses, 2-10 doses, 1-10 doses, or 1-3 doses, over a time period of of 1, 2, 3-6, 6-12, 2-12, 2-20, 3-20, or 4-20 weeks; or, in other embodiments, 2 3, 4, 5, 6, 7, 8, 9, 10, 11, or 12 months; or, in other embodiments, 1.5, 2, 3, 4, 5 years, or more.

Toxicity and therapeutic efficacy of the active ingredients described herein can be determined by standard pharmaceutical procedures in vitro, in cell cultures or experimental animals.

In certain embodiments, the described pharmaceutical composition contains between 100-600 million ASC, for an adult subject. In other embodiments, the pharmaceutical composition contains between 100-400 million, 100-500 million, 150-600 million, 150-500 million, 150-400 million, 200-600 million, 200-500 million, or 200-400 million ASC, for an adult subject. In still other embodiments, the composition contains between 1.5-6 million ASC per kilogram, e.g., for a pediatric subject. In yet other embodiments, e.g., for a pediatric subject, the composition contains between 1.5-5 million, 1.5-4 million, 2-5 million, 2-4 million, 3-6 million, or 3-5 million ASC per kilogram. In certain embodiments, the administration is intramuscular. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from 2 days to 3 weeks or, in other embodiments, from 3 weeks to 3 months, or, in other embodiments, until alleviation of the disease state is achieved.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

Formulations

In some embodiments, the described composition is an injectable composition that is manufactured by adding 1 or more excipients, e.g., stabilizers and aqueous buffers, to placental ASC or CM thereof.

In other embodiments, the ASC are washed to remove serum present therewith. In more specific embodiments, the xenogenic serum components may be reduced by at least 90%, 95%, 99%, 99.5%, 99.8%, or 99.9%, or, in other embodiments, may be undetectable by standard methods, e.g. mass spectrometry.

Pharmaceutical Carriers

In certain embodiments, the described compositions comprise at least one pharmaceutically acceptable carrier. Herein, the term “pharmaceutically acceptable carrier” may refer to a carrier. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are saline and other aqueous carriers, e.g., carriers within 0.1 osmoles per liter (Osm/L) of 0.3 Osm/L.

In other embodiments, the composition further comprises at least one constituent to facilitate formulation, stability, and/or topical application of the composition. In more specific embodiments, the constituent comprises a flow regulating agent, a filler, an excipient, an alcohol, a preservative, a suspending agent, a stabilizer, an aqueous phase, a humectant, or a thickener. In other embodiments, the at least one additional constituent comprises colloidal silica, titanium dioxide, isopropyl alcohol, benzalkonium chloride, stearic acid, cetyl alcohol, isopropyl palmitate, methyparaben, propylparaben, sorbitan monostearate, sorbitol, polysorbate, milk, coconut oil, almond oil, lanolin, lecithin, or beeswax. In other embodiments, the described composition is a gel. In other embodiments, the composition is a lotion.

In still other embodiments, the composition comprises placental ASC in combination with an excipient selected from an osmoprotectant or cryoprotectant, an agent that protects cells from the damaging effect of freezing and ice formation. In certain embodiments, the cryoprotectant is a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. The cells may be any embodiment of ASC mentioned herein, each of which is considered a separate embodiment. In more specific embodiments, DMSO is present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g. human serum albumin (HSA). In certain embodiments, HSA is present at 2-10%, 3-10%, 4-10%, 5-10%, 2-9%, 2-8%, 3-7%, 4-6%, 4.5-5.5%, or 5% (weight per volume). In still other embodiments, DMSO and HSA are both present in a saline solution (a non-limiting example of which is Plasma-Lyte® A (commercially available from Baxter).

In other embodiments, for injection, the described ASC or other active ingredients may be formulated in aqueous solutions, e.g. in a physiologically compatible buffer, non-limiting examples of which are Hank's solution, Ringer's solution, and a physiological salt buffer.

Routes

One may, in various embodiments, administer the described pharmaceutical compositions intramuscularly. In other embodiments, the composition is administered in a systemic manner. Alternatively, one may administer the pharmaceutical composition locally, for example, via injection of the pharmaceutical composition directly into an exposed or affected tissue region of a patient. In other embodiments, the cells are administered intravenously (IV), subcutaneously (SC), or intraperitoneally (IP), each of which is considered a separate embodiment. In this regard, “intramuscular” administration refers to administration into the muscle tissue of a subject; “subcutaneous” to administration just below the skin; “intravenous” to administration into a vein of a subject; and “intraperitoneal” refers to administration into the peritoneum of a subject. In still other embodiments, the cells are administered intratracheally, intrathecally, by inhalation, or intranasally. In certain embodiments, lung-targeting routes of administration are used. In certain embodiments, such routes utilize cells encapsulated in liposomes (or, in some embodiments, other physical barriers) to reduce entrapment within the lungs.

In various embodiments, the described ASC are administered to the subject within 1 hour, within 2 hours, within 3 hours, within 4 hours, within 6 hours, within 8 hours, within 10 hours, within 12 hours, within 15 hours, within 18 hours, within 24 hours, within 30 hours, within 36 hours, within 48 hours, within 3 days, within 4 days, within 5 days, within 6 days, within 8 days, within 10 days, within 12 days, or within 20 days of diagnosis (or, in other embodiments, onset) of any of the herein-described conditions (each of which represents a separate embodiment.). In more specific embodiments, the described compositions are administered 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18-72, 24-72, or 36-72 hours after onset of any of the herein-described conditions. In still other embodiments, the described compositions are administered 3-48, 4-48, 5-48, or 6-48 hours after an onset of any of the herein-described conditions.

In various embodiments, engraftment of the described cells in the host is not required for the cells to exert the described therapeutic effects, each of which is considered a separate embodiment. In other embodiments, engraftment is required for the cells to exert the effect(s). For example, the cells may, in various embodiments, be able to exert a therapeutic effect, without themselves surviving for more than 3 days, more than 4 days, more than 5 days, more than 6 days, more than 7 days, more than 8 days, more than 9 days, more than 10 days, or more than 14 days after administration.

Compositions including the described preparations formulated in a compatible pharmaceutical carrier may also be prepared, placed in an appropriate container, and labeled for treatment of an indicated condition.

It is clarified that each embodiment of the described ASC or CM may be freely combined with each embodiment relating to a therapeutic method or pharmaceutical composition.

Subjects

In certain embodiments, the subject treated by the described methods and compositions is a human. In certain embodiments, the subject has a viral infection. In still other embodiments, the subject has a complication of a viral infection. In some embodiments, the subject is male. In other embodiments, the subject is female. In some embodiments, the subject is at increased risk of complications of viral infection. In certain embodiments, the subject is an elderly subject, for example a subject over 75, or in other embodiments, over 65, over 70, 75, over 80, 70-85, 75-85, or 75-90 years in age. In other embodiments, the subject has asthma. In still other embodiments, the subject has chronic lung disease. In yet other embodiments, the subject has heart disease. In yet other embodiments, the subject is immunocompromised. In yet other embodiments, the subject has chronic kidney disease and/or is undergoing dialysis. In yet other embodiments, the subject has liver disease. In other embodiments, the subject is severely obese (e.g., has a body mass index [BMI] of 40 or higher). In other embodiments, the subject is predisposed to hypercytokinemia, SIRS, or another type of hyperactive immune response.

In yet other embodiments, the subject is a pediatric subject, for example a subject under 18, under 15, under 12, under 10, under 8, under 6, under 5, under 4, under 3, or under 2 years, or under 18, 15, 12, 10, 8, 6, 5, 4, 3, 2, or 1 month in age; or is an adult subject, for example ages 18-60, 18-55, 18-50, 20-60, 20-55, 20-50, 20-45, 20-40, 20-35, 20-30, 25-60, 30-60, 40-60, or 50-60.

In other embodiments, the subject is an animal. In some embodiments, treated animals include domesticated animals and laboratory animals, e.g., non-mammals and mammals, for example non-human primates, rodents, pigs, dogs, and cats. In certain embodiments, the subject is administered with additional therapeutic agents or cells.

Sequential Administration of ASC from Multiple Donors

In certain embodiments, the subject is administered: (a) a first pharmaceutical composition, comprising allogeneic placental ASC from a first donor; and subsequently (b) a second pharmaceutical composition comprising allogeneic placental ASC from a second donor, wherein the second donor differs from the first donor in at least one allele group of HLA-A or HLA-B. In certain embodiments, each of the pharmaceutical compositions contains between 100-600 million ASC, for an adult subject. In other embodiments, the pharmaceutical compositions each contain between 100-400 million, 100-500 million, 150-600 million, 150-500 million, 150-400 million, 200-600 million, 200-500 million, or 200-400 million ASC, for an adult subject. In still other embodiments, the compositions each contain between 1.5-6 million ASC per kilogram, e.g., for a pediatric subject. In yet other embodiments, e.g., for a pediatric subject, the compositions each contain between 1.5-5 million, 1.5-4 million, 2-5 million, 2-4 million, 3-6 million, or 3-5 million ASC per kilogram. In certain embodiments, the administration is intramuscular.

Reference to ASC “from” or “derived from” a donor is intended to encompass cells removed from or otherwise obtained from the donor, followed by optional steps of ex-vivo cell culture, expansion, and/or other treatments to improve the therapeutic efficacy of the cells; and/or combination with pharmaceutical excipients. Those skilled in the art will appreciate that the aforementioned optional steps will not alter the HLA genotype of the ASC, absent intentional modification of the HLA genotype (e.g., using CRISPR-mediating editing or the like). Cell populations with an intentionally modified HLA genotype are not intended to be encompassed. ASC populations that contain a mixture of cells from more than one donor are also not intended to be encompassed.

As will be appreciated by those skilled in the art, the HLA system or complex is a gene complex encoding the major histocompatibility complex (MHC) proteins in humans. These cell-surface proteins are involved in regulation of the immune system in humans. The HLA gene complex resides on a 3-Mbp stretch within chromosome 6p21. HLA genes are highly polymorphic. HLAs encoding MHC class I proteins (“class I HLA's”) present peptides from inside the cell, while class II HLA's present external peptides.

There are 3 major MHC class I genes, HLA-A, HLA-B, and HLA-C; and 3 minor class I genes, HLA-E, HLA-F and HLA-G. β2-microglobulin binds with major and minor gene subunits to produce a heterodimer.

There are 3 major (DP, DQ and DR) and 2 minor (DM and DO) MHC class II proteins encoded by the HLA. The class H MHC proteins combine to form heterodimeric (αβ) protein receptors that are typically expressed on the surface of antigen-presenting cells.

HLA alleles are often named according to a multi-partite system, where the letter prefix (e.g. “HLA-A”) denotes the locus, followed by an asterisk; followed by the “allele group” number; followed by the specific HLA protein number; followed by a number used to denote silent DNA mutations in a coding region; followed by, lastly, a number used to denote DNA mutations in a non-coding region (Robinson J el al.). For example, in the hypothetical allele “HLA-A*02:07:01:03”, the allele group number is 02; 07 is the specific HLA protein number; 01 describes a pattern of silent DNA mutations in the coding regions; and 03 describes a pattern of DNA mutations in non-coding regions. “Mutations” in this regard refers to variations relative to the founder (initially identified) allele in the allele group. Typically, an allele group corresponds to a particular encoded serological antigen, while specific HLA proteins within an allele group exhibit relatively minor differences. In certain embodiments, an “antigenic” difference refers to a different allele group, while an “allelic” difference refers to a different HLA protein within the same allele group.

Allele groups are clustered into “supertypes” which have similar peptide binding repertoires. Examples of HLA-A supertypes are 1, 2, 3, and 24, and examples of HLA-B supertypes are 7, 27, 44, 58, and 62. Typically, an allele supertype corresponds to a particular encoded serological antigen.

Reference to a second donor “differ/differs/differing” from a first donor in at least one allele group of HLA-A or HLA-B denotes that the DNA of the second donor comprises at least one HLA-A or HLA-B allele belonging to an allele group not represented in the alleles of the first donor. (Typically [except in the case of a homozygous first donor], the DNA of the first donor will also comprise at least one HLA-A or HLA-B allele belonging to an allele group not represented in the alleles of the second donor). Similarly, a second donor “differs from” a first donor in at least one allele supertype if the DNA of the second donor comprises at least one HLA-A or HLA-B allele belonging to a supertype not represented in the alleles of the first donor. These terms are intended to be used analogously in various contexts herein, except where indicated otherwise.

In other embodiments, the second donor in the described therapeutic methods and compositions differs from the first donor in at least one allele group of HLA-A. In still other embodiments, the second donor differs from the first donor in at least one allele group of HLA-B.

In yet other embodiments, the second donor differs from the first donor in at least two HLA-A allele groups of or, in other embodiments, in at least 2 HLA-B allele groups; or, in other embodiments, at least one allele group of each of HLA-A and HLA-B.

In other embodiments, the second donor differs from the first donor in at least one HLA-A allele supertype or, in other embodiments, at least one HLA-B allele supertype.

In still other embodiments, the second donor differs from the first donor in at least two allele supertypes of HLA-A or HLA-B, which may be, in more specific embodiments, an HLA-A allele supertype, an HLA-B allele supertype, or a combination thereof.

Alternatively or in addition, the second donor differs from the first donor in at least one allele group of HLA-DR, or in other embodiments, in 2 HLA-DR allele groups.

Step b) of the described method (administering a second pharmaceutical composition comprising allogeneic ASC from a second donor) is, in various embodiments, performed between 2-52 weeks after step (a). In other embodiments, step b) is performed between 3-52, 4-26, 5-26, 6-20, 6-18, 6-15, 6-10, 3-20, 3-15, 3-10, 4-12, 4-20, 5-18, 6-16, 8-16, 10-16, or 8-12 weeks after step a).

Alternatively or in addition, step b) of the described methods is followed by an additional step, comprising the step of administering to the subject, at least 7 days after step b), a third pharmaceutical composition comprising allogeneic ASC derived from a third donor, wherein the third donor differs from both the first donor and the second donor in at least one allele group of HLA-A or HLA-B (i.e. has an allele group not represented in either the first or second donor), which is, in various embodiments, an allele of HLA-A or HLA-B. In other embodiments, the third donor differs from both the first donor and the second donor in at least two allele groups of HLA-A or HLA-B, which are, in various embodiments, an allele of HLA-A, HLA-B, or a combination thereof.

Further embodiments of dosing regimens are described in WO 2019/239295, in the name of Zami Aberman et al., which is incorporated herein by reference.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternal tissue-derived cells were prepared as described in Example 1 of International Patent Application WO 2016/098061, which is incorporated herein by reference in its entirety. The cell expansion and harvesting process consisted of 3 stages, followed by downstream processing steps: Stage 1, the intermediate cell stock (ICS) production; Stage 2, the thawing of the ICS and initial further culture steps; and Stage 3, additional culture steps, first in tissue culture dishes, and then on Fibra-Cel® carriers in a bioreactor. All steps were performed in the presence of serum-containing medium. The downstream processing steps included harvest from flasks or bioreactor/s, cell concentration, washing, formulation, filling and cryopreservation. The procedure included periodic testing of the growth medium for sterility and contamination, all as described in international patent application publ. no. WO 2019/239295, which is incorporated herein by reference.

### Example 2: Culture of Placental Ceils in Serum-Free Medium

Methods

The cell harvesting and expansion process consisted of 3 stages, followed by downstream processing steps: Stage 1 the intermediate cell stock (ICS) production; Stage 2, the thawing of the ICS and initial further culture steps; and Stage 3, additional culture steps, first in tissue culture dishes, and then on Fibra-Cel® carriers in a bioreactor. The downstream processing steps included harvest from flasks or bioreactor/s, cell concentration, washing, formulation, filling and cryopreservation. The procedure included periodic testing of the growth medium for sterility and contamination, all as described in international patent application publ. no. WO 2019/239295, which is incorporated herein by reference. Bone marrow migration assays were also performed as described in WO 2019/239295.

Results

Placental cells were extracted and expanded in serum-free (SF) medium for 3 passages. Cell characteristics of eight batches were assessed and were found to exhibit similar patterns of cell size and PDL (population doubling level since passage 1) as shown for a representative batch in Table 1. Cells also significantly enhanced hematopoiesis in a bone marrow migration (BMM) assay.

TABLE 1
Characteristics of placental cells expanded in SF medium.

| | | | Total | cell | |
|---|---|---|---|---|---|
| | | | growth | size | |
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Osteocyte and Adipocyte Differentiation Assays

ASC were prepared as described in Example 1. BM adherent cells were obtained as described in WO 2016/098061 to Esther Lukasiewicz Hagai and Rachel Ofir, which is incorporated herein by reference in its entirety. Osteogenesis and adipogenesis assays were performed as described in WO 2016/098061.

Osteocyte induction. Incubation of BM-derived adherent cells in osteogenic induction medium resulted in differentiation of over 50% of the BM cells, as demonstrated by positive alizarin red staining. On the contrary, none of the placental-derived cells exhibited signs of osteogenic differentiation.

Next, a modified osteogenic medium comprising Vitamin D and higher concentrations of dexamethasone was used. Over 50% of the BM cells underwent differentiation into osteocytes, while none of the placental-derived cells exhibited signs of osteogenic differentiation.

Adipocyte induction. Adipocyte differentiation of placenta- or BM-derived adherent cells in adipocyte induction medium resulted in differentiation of over 50% of the BM-derived cells, as demonstrated by positive oil red staining and by typical morphological changes (e.g. accumulation of oil droplets in the cytoplasm). In contrast, none of the placental-derived cells differentiated into adipocytes.

Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 4: Further Osteocyte and Adipocyte Differentiation Assays

ASC were prepared as described in Example 2. Adipogenesis and Osteogenesis were assessed using the STEMPRO® Adipogenesis Differentiation Kit (GIBCO, Cat #A1007001) and the STEMPRO® Osteogenesis Differentiation Kit (GIBCO, Cat #A1007201), respectively.

Results

Adipogenesis and Osteogenesis of placental cells grown in SRM or in full DMEM were tested. Groups are shown in Table 2.

TABLE 2
experimental groups

| Group | Product | Batch |
|---|---|---|
| A1 | BM derived MSC | BM-122 |
| | (positive control) | |
| B1 | ASC grown in SRM | PD220914SFMS3 R001 B1.2 |
| C1 | ASC grown in SRM | R050115 R01 |
| D1 | ASC grown in SRM | R280115 R01 |
| E1 | ASC grown in full DMEM | PT041011R36 |

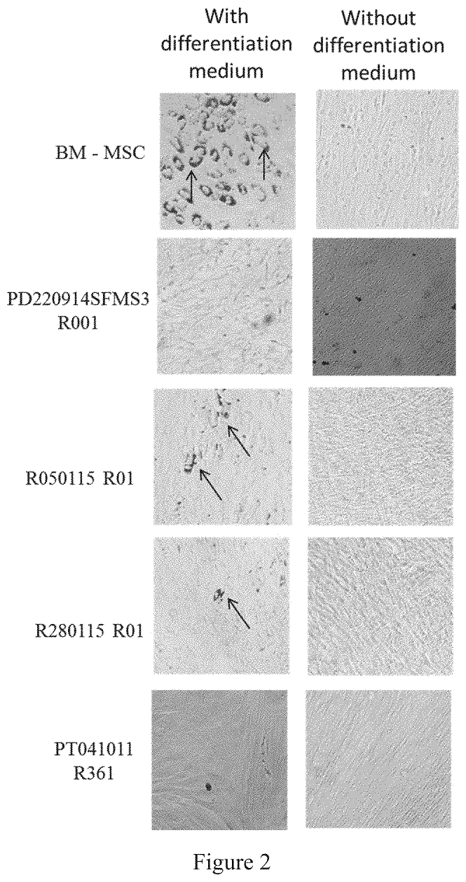

In adipogenesis assays, BM-MSCs treated with differentiation medium stained positively with Oil Red O (FIG. 2). By contrast, ⅔ of the SRM batches exhibited negligible staining, and the other SRM batch, as well as the full DMEM-grown cells, did not exhibit any staining at all, showing that they lacked significant adipogenic potential.

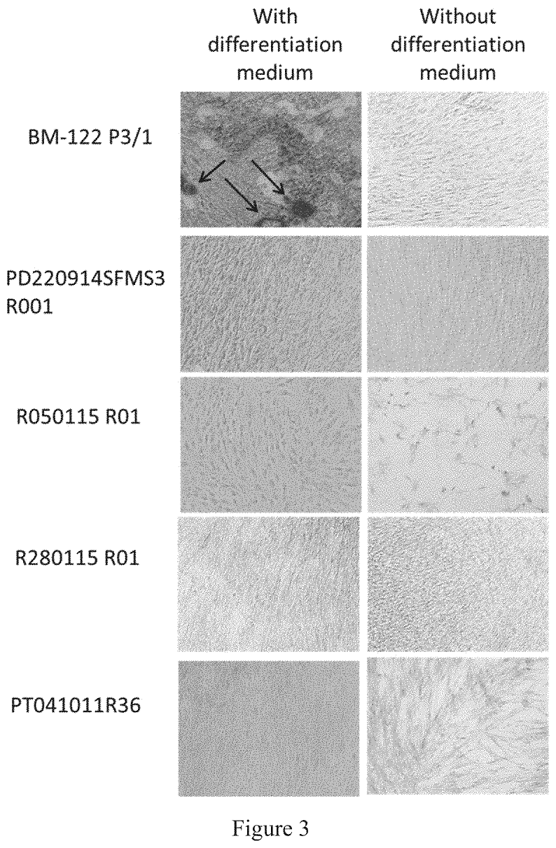

In osteogenesis assays, BM-MSCs treated with differentiation medium stained positively with Alizarin Red S (FIG. 3). By contrast, none of the placental cell batches grown in SRM or full DMEM exhibited staining, showing that they lacked significant osteogenic potential.

### Example 5. Studies of Factors Secreted by Placental ASC

CM was prepared from two batches each of maternal ASC, fetal ASC expanded in serum-containing medium, and fetal ASC expanded in SFM, after a 6-day bioreactor incubation; or a 2-day incubation in plates, changing the medium once per day.

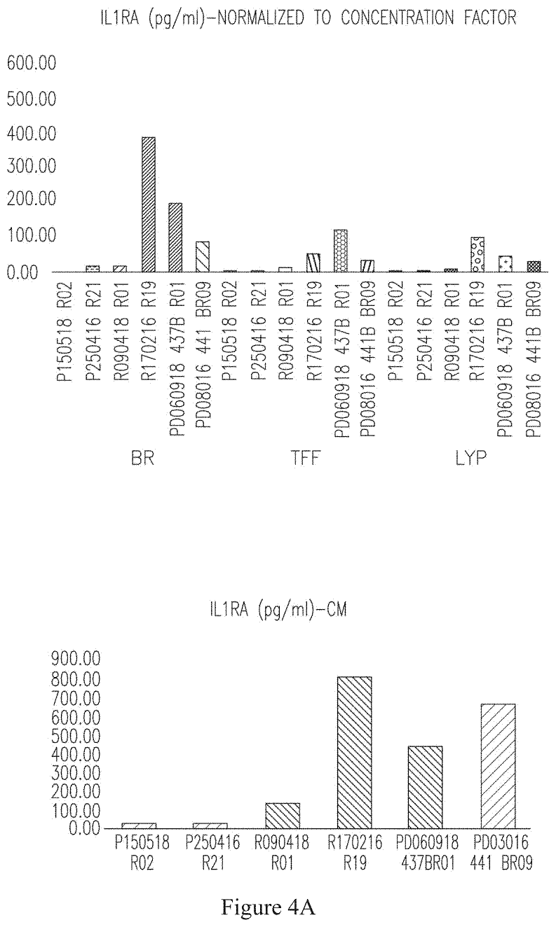

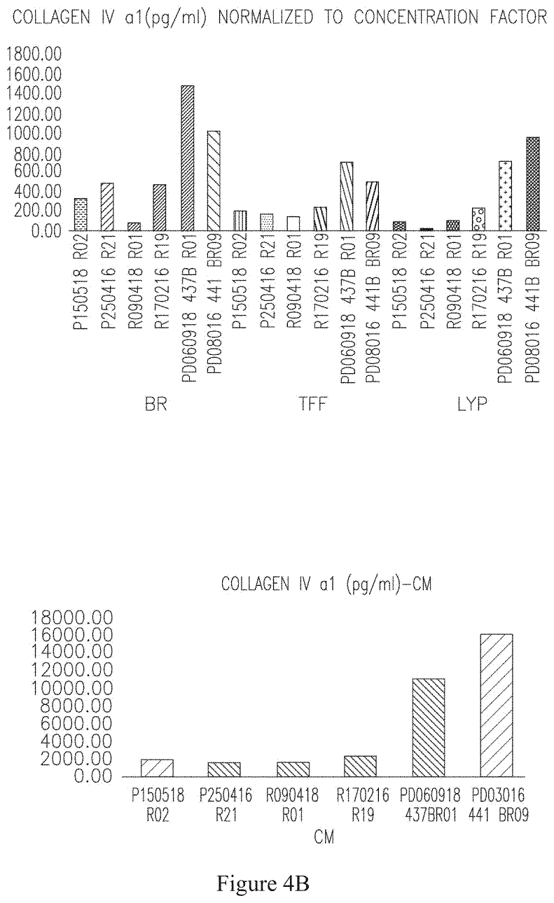

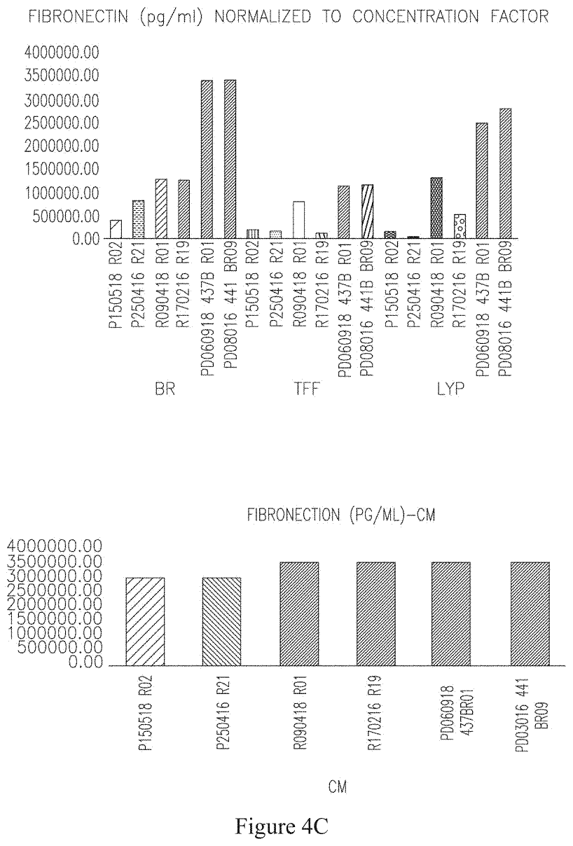

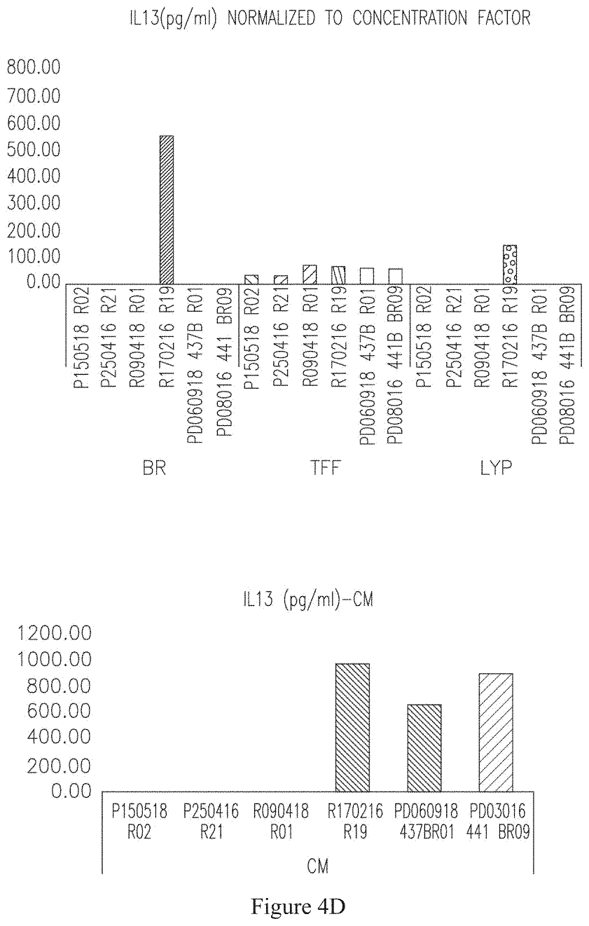

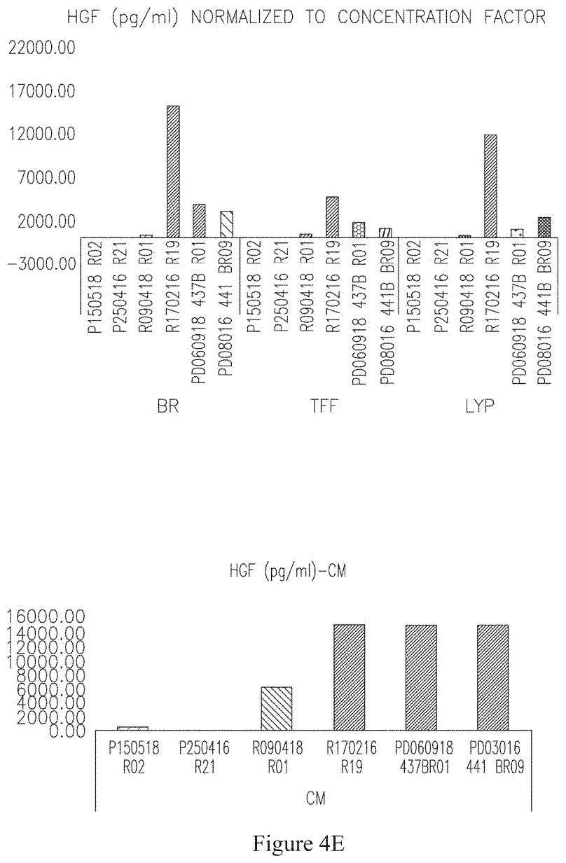

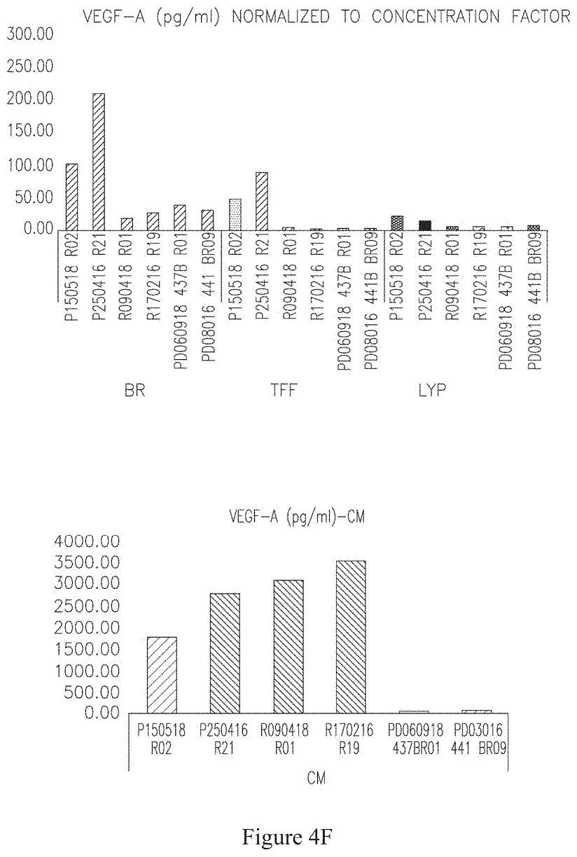

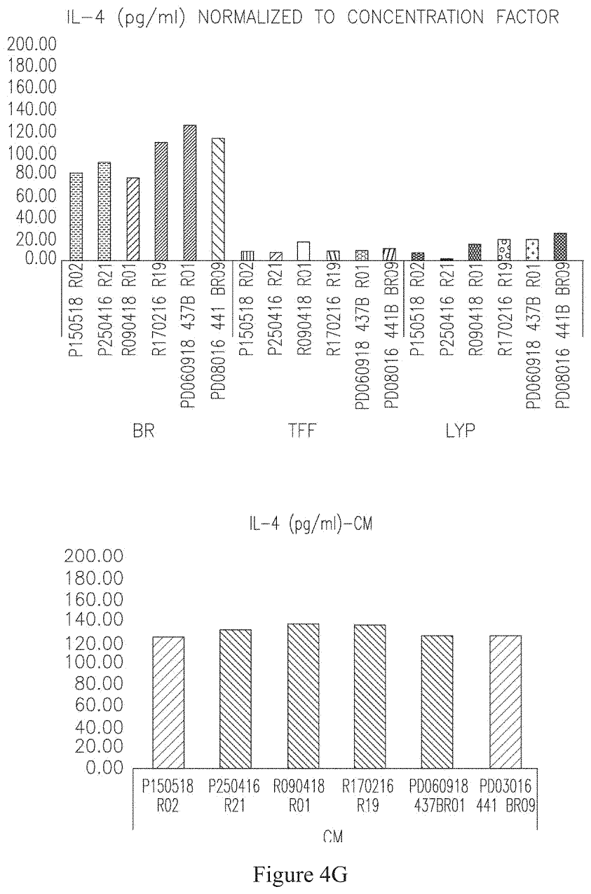

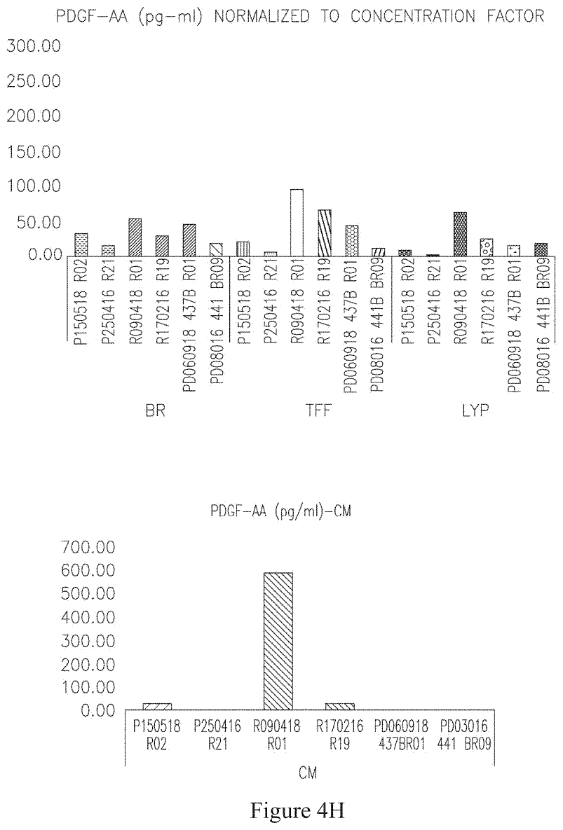

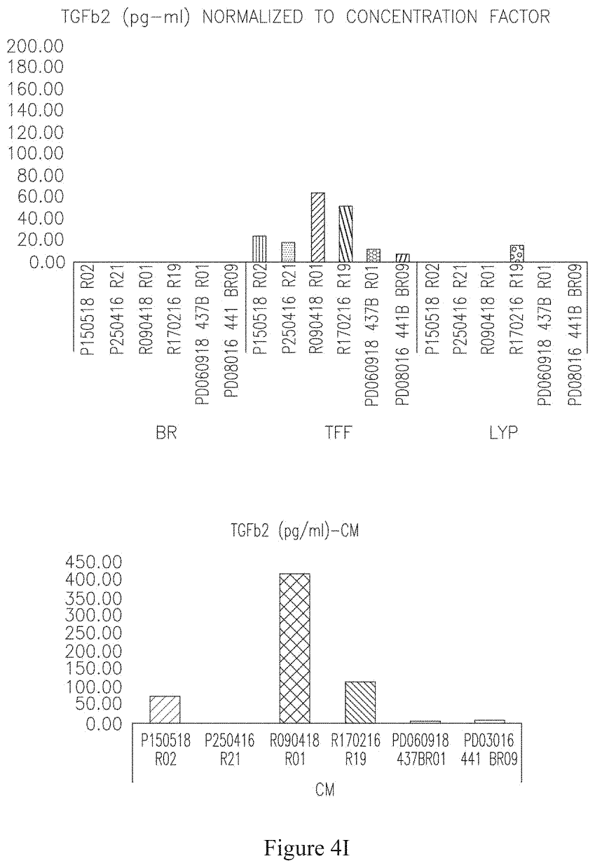

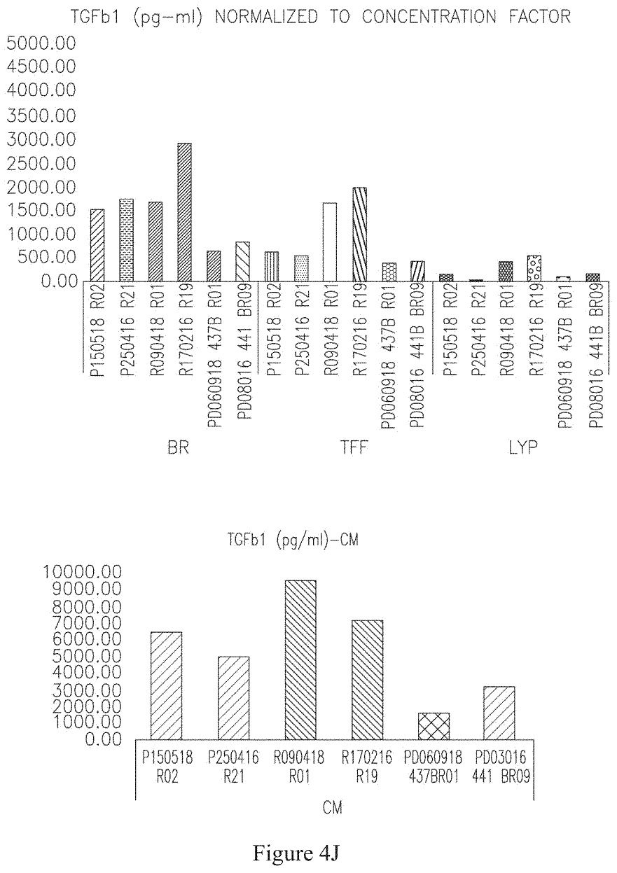

Secreted protein expression was measured by Luminex®. Collagen 1-alpha were highly expressed in all samples. IL-1-ra, Collagen TV-1a, Fibronectin, IL-13, HGF, VEGF-A, IL-4, PDGF-AA, TIMP-1, TGFb2, TGFb1 were all significantly expressed in at least some samples, while IL-16 was expressed at negligible or no level (FIGS. 4A-J and Tables 3-4).

TABLE 3
summarizes protein expression of the indicated
proteins in bioreactor media.

| Protein | Maternal | Fetal/serum | Fetal/SF |
|---|---|---|---|
| Collagen 1a | +++ | +++ | +++ |
| IL-10 | − | − | − |
| EGF | − | − | − |
| IL-1RA | − | ++ | ++ |
| bFGF | − | − | ++ |
| Collagen IVa1 | ++ | ++ | +++ |
| Fibronectin | +++ | +++ | +++ |
| IL-13 | + | ++ | + |
| HGF | − | +++ | +++ |
| MMP-1 | +++ | +++ | +++ |
| MMP-2 | +++ | +++ | +++ |
| IL-16 | + | + | + |
| VEGF-A | ++ | + | + |
| IL-4 | + | + | + |
| PDGF-AA | + | + | + |
| TIMP1 | +++ | +++ | +++ |
| TGFb3 | − | − | − |
| TGFb2 | + | + | + |
| TGFb1 | +++ | +++ | +++ |

−, +, ++, +++ indicate <10, 10-100, 100-1000, and >1000 pg/ml, respectively.

Mass spectrometry was performed on fetal/placental ASC-CM from a bioreactor incubation, and tryptic peptides of human origin were identified by their sequences. The peptides are shown in Table 4.

TABLE 4
Tryptic peptides from placental ASC-CM. “HS” refers to Homo sapiens.

| Protein name | gene name (indicated after “GN”) | Uniprot name (in square brackets) |
|---|---|---|
| z | z | z |

Alpha-2-macroglobulin OS = HS GN = A2M PE = 1 SV = 3 − [A2MG_HUMAN]
Agrin OS = HS GN = AGRN PE = 1 SV = 5 − [AGRIN_HUMAN]
Serum albumin OS = HS GN = ALB PE = 1 SV = 1 − [A0A0C4DGB6_HUMAN]
Annexin A1 OS = HS GN = ANXA1 PE = 1 SV = 2 − [ANXA1_HUMAN]
Annexin (Fragment) OS = HS GN = ANXA2 PE = 1 SV = 1 − [H0YMM1_HUMAN]
APOC2 protein OS = HS GN = APOC2 PE = 1 SV = 1 − [Q6P163_HUMAN]
Actin-related protein 2/3 complex subunit 2 OS = HS GN = ARPC2 PE = 1 SV = 1 −
[ARPC2_HUMAN]
Renin receptor (Fragment) OS = HS GN = ATP6AP2 PE = 1 SV = 1 − [A0A1B0GWD6_HUMAN]
Beta-2-microglobulin (Fragment) OS = HS GN = B2M PE = 1 SV = 1 − [H0YLF3_HUMAN]
Beta-1,4-glucuronyltransferase 1 OS = HS GN = B4GAT1 PE = 1 SV = 1 − [B4GA1_HUMAN]
Bone morphogenetic protein 1 OS = HS GN = BMP1 PE = 1 SV = 2 − [BMP1_HUMAN]
Complement C4-A OS = HS GN = C4A PE = 1 SV = 1 − [A0A0G2JPR0_HUMAN]
Calcium-binding protein 39-like OS = HS GN = CAB39L PE = 1 SV = 1 − [B7ZBJ4_HUMAN]
Cell adhesion molecule 1 OS = HS GN = CADM1 PE = 1 SV = 1 − [A0A087X0T8_HUMAN]
Capping protein (Actin filament) muscle Z-line, beta, isoform CRA_a OS = HS GN = CAPZB PE = 1
SV = 1 − [B1AK87_HUMAN]
CD44 antigen OS = HS GN = CD44 PE = 1 SV = 2 − [H0YD13_HUMAN]
Tetraspanin OS = HS GN = CD81 PE = 1 SV = 1 − [E9PJK1_HUMAN]
Cadherin-2 OS = HS GN = CDH2 PE = 1 SV = 4 − [CADH2_HUMAN]
Chymotrypsin-like elastase family member 1 OS = HS GN = CELA1 PE = 1 SV = 2 −
[CELA1_HUMAN]
Collagen alpha-1(XI) chain (Fragment) OS = HS GN = COL11A1 PE = 1 SV = 8 −
[C9JMN2_HUMAN]
Collagen alpha-1(XII) chain OS = HS GN = COL12A1 PE = 1 SV = 1 − [D6RGG3_HUMAN]
Collagen alpha-1(I) chain OS = HS GN = COL1A1 PE = 1 SV = 5 − [CO1A1_HUMAN]
Collagen alpha-1(III) chain OS = HS GN = COL3A1 PE = 1 SV = 4 − [CO3A1_HUMAN]
Collagen alpha-1(IV) chain OS = HS GN = COL4A1 PE = 1 SV = 3 − [CO4A1_HUMAN]
Collagen alpha-2(IV) chain OS = HS GN = COL4A2 PE = 1 SV = 4 − [CO4A2_HUMAN]
Collagen alpha-1(VI) chain OS = HS GN = COL6A1 PE = 1 SV = 1 − [A0A087X0S5_HUMAN]
Collagen alpha-3(VI) chain OS = HS GN = COL6A3 PE = 1 SV = 5 − [CO6A3_HUMAN]
Ceruloplasmin OS = HS GN = CP PE = 1 SV = 1 − [CERU_HUMAN]
Cystatin-C OS = HS GN = CST3 PE = 1 SV = 1 − [CYTC_HUMAN]
Connective tissue growth factor OS = HS GN = CTGF PE = 1 SV = 2 − [CTGF_HUMAN]
Cathepsin Z OS = HS GN = CTSZ PE = 1 SV = 1 − [CATZ_HUMAN]
Protein CutA OS = HS GN = CUTA PE = 1 SV = 1 − [C9IZG4_HUMAN]
Stromal cell-derived factor 1 OS = HS GN = CXCL12 PE = 1 SV = 1 − [SDF1_HUMAN]
Cytoplasmic FMR1-interacting protein 1 OS = HS GN = CYFIP1 PE = 1 SV = 1 − [CYFP1_HUMAN]
Protein CYR61 OS = HS GN = CYR61 PE = 1 SV = 1 − [CYR61_HUMAN]
Dermcidin OS = HS GN = DCD PE = 1 SV = 2 − [DCD_HUMAN]
Dickkopf-related protein 1 OS = HS GN = DKK1 PE = 1 SV = 1 − [DKK1_HUMAN]
Desmoglein-1 OS = HS GN = DSG1 PE = 1 SV = 2 − [DSG1_HUMAN]
Desmoplakin OS = HS GN = DSP PE = 1 SV = 3 − [DESP_HUMAN]
EF-hand domain-containing protein D2 OS = HS GN = EFHD2 PE = 1 SV = 1 − [EFHD2_HUMAN]
Eukaryotic translation initiation factor 4 gamma 1 (Fragment) OS = HS GN = EIF4G1 PE = 1 SV = 1 −
[C9J6B6_HUMAN]
Eukaryotic translation initiation factor 5A OS = HS GN = EIF5A2 PE = 1 SV = 1 −
[F8WCJ1_HUMAN]
Fatty acid-binding protein, heart OS = HS GN = FABP3 PE = 1 SV = 1 − [S4R3A2_HUMAN]
Fibulin-1 OS = HS GN = FBLN1 PE = 1 SV = 1 − [B1AHL2_HUMAN]
Fibrillin-1 OS = HS GN = FBN1 PE = 1 SV = 3 − [FBN1_HUMAN]
Filamin-A OS = HS GN = FLNA PE = 1 SV = 1 − [Q5HY54_HUMAN]
Fibronectin OS = HS GN = FN1 PE = 1 SV = 4 − [FINC_HUMAN]
Follistatin-related protein 1 OS = HS GN = FSTL1 PE = 1 SV = 1 − [FSTL1_HUMAN]
Rab GDP dissociation inhibitor beta OS = HS GN = GDI2 PE = 1 SV = 2 − [GDIB_HUMAN]
Glypican-1 OS = HS GN = GPC1 PE = 1 SV = 2 − [H7C410_HUMAN]
Histone H3 OS = HS GN = H3F3B PE = 1 SV = 1 − [K7EMV3_HUMAN]
HCG1745306, isoform CRA_a OS = HS GN = HBA2 PE = 1 SV = 1 − [G3V1N2_HUMAN]
Hemoglobin subunit delta OS = HS GN = HBD PE = 1 SV = 2 − [HBD_HUMAN]
Hepatocyte growth factor activator OS = HS GN = HGFAC PE = 1 SV = 1 − [HGFA_HUMAN]
Histone H2A type 1-H OS = HS GN = HIST1H2AH PE = 1 SV = 3 − [H2A1H_HUMAN]
HLA class I histocompatibility antigen, Cw-6 alpha chain OS = HS GN = HLA-C PE = 1 SV = 1 −
[A0A140T9Z4_HUMAN]
Heterogeneous nuclear ribonucleoproteins A2/B1 OS = HS GN = HNRNPA2B1 PE = 1 SV = 1 −
[A0A087WUI2_HUMAN]
Hornerin OS = HS GN = HRNR PE = 1 SV = 2 − [HORN_HUMAN]
Heat shock protein HSP 90-alpha OS = HS GN = HSP90AA1 PE = 1 SV = 5 − [HS90A_HUMAN]
Endoplasmin OS = HS GN = HSP90B1 PE = 1 SV = 1 − [Q96GW1_HUMAN]
Heat shock 70 kDa protein 1B OS = HS GN = HSPA1B PE = 1 SV = 1 − [HS71B_HUMAN]
Heat shock cognate 71 kDa protein OS = HS GN = HSPA8 PE = 1 SV = 1 − [E9PKE3_HUMAN]
Basement membrane-specific heparan sulfate proteoglycan core protein OS = HS GN = HSPG2
PE = 1 SV = 4 − [PGBM_HUMAN]
Serine protease HTRA1 OS = HS GN = HTRA1 PE = 1 SV = 1 − [HTRA1_HUMAN]
E3 ubiquitin-protein ligase HUWE1 OS = HS GN = HUWE1 PE = 1 SV = 3 − [HUWE1_HUMAN]
Insulin-like growth factor-binding protein 5 OS = HS GN = IGFBP5 PE = 1 SV = 1 − [IBP5_HUMAN]
Insulin-like growth factor-binding protein 6 OS = HS GN = IGFBP6 PE = 1 SV = 1 − [IBP6_HUMAN]
Insulin-like growth factor-binding protein 7 OS = HS GN = IGFBP7 PE = 1 SV = 1 − [IBP7_HUMAN]
Insulin-like growth factor I (Fragment) OS = HS GN = IGF-I PE = 1 SV = 1 − [Q13429_HUMAN]
Junction plakoglobin OS = HS GN = JUP PE = 1 SV = 3 − [PLAK_HUMAN]
Keratinocyte proline-rich protein OS = HS GN = KPRP PE = 1 SV = 1 − [KPRP_HUMAN]
Laminin subunit alpha-1 OS = HS GN = LAMA1 PE = 1 SV = 2 − [LAMA1_HUMAN]
Laminin subunit alpha-4 OS = HS GN = LAMA4 PE = 1 SV = 1 − [A0A0A0MTC7_HUMAN]
Laminin subunit beta-1 OS = HS GN = LAMB1 PE = 1 SV = 2 − [LAMB1_HUMAN]
Laminin subunit gamma-1 OS = HS GN = LAMC1 PE = 1 SV = 3 − [LAMC1_HUMAN]
Galectin-1 OS = HS GN = LGALS1 PE = 1 SV = 2 − [LEG1_HUMAN]
Galectin-3 OS = HS GN = LGALS3 PE = 1 SV = 5 − [LEG3_HUMAN]
Galectin-3-binding protein OS = HS GN = LGALS3BP PE = 1 SV = 1 − [LG3BP_HUMAN]
LIM and senescent cell antigen-like-containing domain protein 1 OS = HS GN = LIMS1 PE = 1 SV = 4 −
[LIMS1_HUMAN]
Vesicular integral-membrane protein VIP36 OS = HS GN = LMAN2 PE = 1 SV = 1 −
[LMAN2_HUMAN]
Protein-lysine 6-oxidase OS = HS GN = LOX PE = 1 SV = 2 − [LYOX_HUMAN]
Lysyl oxidase homolog 2 (Fragment) OS = HS GN = LOXL2 PE = 1 SV = 1 − [H0YAR1_HUMAN]
Latent-transforming growth factor beta-binding protein 2 OS = HS GN = LTBP2 PE = 1 SV = 1 −
[G3V3X5_HUMAN]
Lysozyme C OS = HS GN = LYZ PE = 1 SV = 1 − [LYSC_HUMAN]
72 kDa type IV collagenase OS = HS GN = MMP2 PE = 1 SV = 2 − [MMP2_HUMAN]
Moesin OS = HS GN = MSN PE = 1 SV = 3 − [MOES_HUMAN]
Metallothionein-1E OS = HS GN = MT1E PE = 1 SV = 1 − [MT1E_HUMAN]
Matrix-remodeling-associated protein 5 OS = HS GN = MXRA5 PE = 2 SV = 3 − [MXRA5_HUMAN]
Myosin-9 OS = HS GN = MYH9 PE = 1 SV = 4 − [MYH9_HUMAN]
Myosin light polypeptide 6 (Fragment) OS = HS GN = MYL6 PE = 1 SV = 1 − [F8VPF3_HUMAN]
Neurobeachin-like protein 2 OS = HS GN = NBEAL2 PE = 1 SV = 2 − [NBEL2_HUMAN]
Nidogen-1 OS = HS GN = NID1 PE = 1 SV = 3 − [NID1_HUMAN]
Epididymal secretory protein E1 (Fragment) OS = HS GN = NPC2 PE = 1 SV = 1 −
[G3V2V8_HUMAN]
Puromycin-sensitive aminopeptidase OS = HS GN = NPEPPS PE = 1 SV = 1 − [E9PLK3_HUMAN]
Nuclear transport factor 2 (Fragment) OS = HS GN = NUTF2 PE = 1 SV = 1 − [H3BRV9_HUMAN]
Ubiquitin thioesterase OTUB1 OS = HS GN = OTUB1 PE = 1 SV = 1 − [F5GYN4_HUMAN]
Beta-parvin OS = HS GN = PARVB PE = 1 SV = 1 − [A0A087WZB5_HUMAN]
Pterin-4-alpha-carbinolamine dehydratase OS = HS GN = PCBD1 PE = 1 SV = 2 − [PHS_HUMAN]
Profilin-1 OS = HS GN = PFN1 PE = 1 SV = 2 − [PROF1_HUMAN]
Profilin OS = HS GN = PFN2 PE = 1 SV = 1 − [C9J712_HUMAN]
Glycerol-3-phosphate phosphatase OS = HS GN = PGP PE = 1 SV = 1 − [PGP_HUMAN]
Fibrocystin-L OS = HS GN = PKHD1L1 PE = 2 SV = 2 − [PKHL1_HUMAN]
Periostin OS = HS GN = POSTN PE = 1 SV = 1 − [B1ALD9_HUMAN]
Ribose-phosphate pyrophosphokinase 3 OS = HS GN = PRPS1L1 PE = 1 SV = 1 −
[A0A0B4J207_HUMAN]
Serine protease 23 (Fragment) OS = HS GN = PRSS23 PE = 1 SV = 1 − [E9PRR2_HUMAN]
Proteasome subunit alpha type-3 OS = HS GN = PSMA3 PE = 1 SV = 2 − [PSA3_HUMAN]
Proteasome subunit alpha type OS = HS GN = PSMA6 PE = 1 SV = 1 − [G3V295_HUMAN]
Proteasome subunit beta type-2 OS = HS GN = PSMB2 PE = 1 SV = 1 − [PSB2_HUMAN]
26S proteasome non-ATPase regulatory subunit 3 OS = HS GN = PSMD3 PE = 1 SV = 2 −
[PSMD3_HUMAN]
26S proteasome non-ATPase regulatory subunit 8 (Fragment) OS = HS GN = PSMD8 PE = 1 SV = 8 −
[K7EJR3_HUMAN]
Prostaglandin-H2 D-isomerase OS = HS GN = PTGDS PE = 1 SV = 1 − [PTGDS_HUMAN]
Peroxidasin homolog OS = HS GN = PXDN PE = 1 SV = 2 − [PXDN_HUMAN]
Sulfhydryl oxidase 1 OS = HS GN = QSOX1 PE = 1 SV = 3 − [QSOX1_HUMAN]
Ras-related protein Rab-11A (Fragment) OS = HS GN = RAB11A PE = 4 SV = 1 −
[H3BMH2_HUMAN]
Ras-related protein Rab-2B OS = HS GN = RAB2B PE = 1 SV = 1 − [E9PE37_HUMAN]
Ras-related protein Rab-5C (Fragment) OS = HS GN = RAB5C PE = 1 SV = 1 − [F8VVK3_HUMAN]
GTP-binding nuclear protein Ran (Fragment) OS = HS GN = RAN PE = 1 SV = 8 −
[F5H018_HUMAN]
Retinoic acid receptor responder protein 2 OS = HS GN = RARRES2 PE = 1 SV = 1 −
[RARR2_HUMAN]
60S acidic ribosomal protein P0 (Fragment) OS = HS GN = RPLP0 PE = 1 SV = 1 −
[F8VPE8_HUMAN]
40S ribosomal protein S2 (Fragment) OS = HS GN = RPS2 PE = 1 SV = 1 − [H0YEN5_HUMAN]
Ras suppressor protein 1 OS = HS GN = RSU1 PE = 1 SV = 3 − [RSU1_HUMAN]
Syndecan-4 OS = HS GN = SDC4 PE = 1 SV = 2 − [SDC4_HUMAN]
Alpha-1-antichymotrypsin OS = HS GN = SERPINA3 PE = 1 SV = 1 − [G3V3A0_HUMAN]
Plasminogen activator inhibitor 1 OS = HS GN = SERPINE1 PE = 1 SV = 1 − [PAI1_HUMAN]
Glia-derived nexin OS = HS GN = SERPINE2 PE = 1 SV = 1 − [GDN_HUMAN]
SH3 domain-binding glutamic acid-rich-like protein 3 OS = HS GN = SH3BGRL3 PE = 1 SV = 1 −
[Q5T123_HUMAN]
Sorbitol dehydrogenase OS = HS GN = SORD PE = 1 SV = 1 − [H0YLA4_HUMAN]
SPARC OS = HS GN = SPARC PE = 1 SV = 1 − [SPRC_HUMAN]
Testican-1 OS = HS GN = SPOCK1 PE = 1 SV = 1 − [TICN1_HUMAN]
Soluble scavenger receptor cysteine-rich domain-containing protein SSC5D OS = HS GN = SSC5D
PE = 1 SV = 3 − [SRCRL_HUMAN]
Stanniocalcin-2 (Fragment) OS = HS GN = STC2 PE = 1 SV = 1 − [H0YB13_HUMAN]
Tissue factor pathway inhibitor 2 OS = HS GN = TFPI2 PE = 1 SV = 1 − [TFPI2_HUMAN]
Thrombospondin-1 OS = HS GN = THBS1 PE = 1 SV = 2 − [TSP1_HUMAN]
Metalloproteinase inhibitor 1 OS = HS GN = TIMP1 PE = 1 SV = 1 − [TIMP1_HUMAN]
Metalloproteinase inhibitor 2 OS = HS GN = TIMP2 PE = 1 SV = 2 − [TIMP2_HUMAN]
Tenascin OS = HS GN = TNC PE = 1 SV = 3 − [TENA_HUMAN]
Tropomyosin alpha-3 chain OS = HS GN = TPM3 PE = 1 SV = 1 − [A0A087WWU8_HUMAN]
Tropomyosin alpha-4 chain OS = HS GN = TPM4 PE = 1 SV = 3 − [TPM4_HUMAN]
Translationally-controlled tumor protein OS = HS GN = TPT1 PE = 1 SV = 1 − [TCTP_HUMAN]
Translin (Fragment) OS = HS GN = TSN PE = 1 SV = 1 − [H7C1D4_HUMAN]
Tubulin beta chain OS = HS GN = TUBB PE = 1 SV = 1 − [Q5JP53_HUMAN]
Polyubiquitin-C (Fragment) OS = HS GN = UBC PE = 1 SV = 1 − [F5GYU3_HUMAN]
Ubiquitin-conjugating enzyme E2 N OS = HS GN = UBE2N PE = 1 SV = 1 − [F8VQQ8_HUMAN]
Versican core protein OS = HS GN = VCAN PE = 1 SV = 3 − [CSPG2_HUMAN]
Vimentin OS = HS GN = VIM PE = 1 SV = 1 − [B0YJC4_HUMAN]
Vacuolar protein sorting-associated protein 29 OS = HS GN = VPS29 PE = 1 SV = 1 −
[VPS29_HUMAN]

### Example 6: Immunogenicity & Immunomodulatory Properties of ASC

The expression of co-stimulatory molecules on ASC was measured. FACS analysis demonstrated the absence of CD80, CD86 and CD40 on the cell membranes. Moreover, the cells expressed low levels HLA class I molecules, as detected by staining for HLA A/B/C.

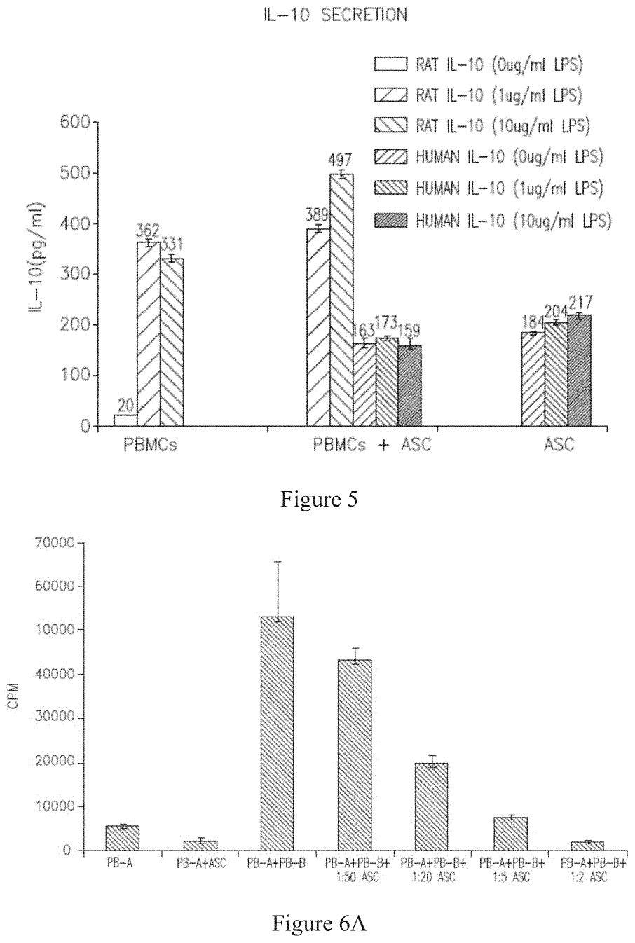

To further investigate the immunogenicity and the immunomodulatory properties of the cells, human/rat Mixed Lymphocyte Reaction (MLR) tests were performed. Rat PBMC were stimulated with LPS (lipopolysaccharide) in the absence or presence of (human) ASC, and secretion of IL-10 by the PBMC was measured. ASC increased the IL-10 secretion (FIG. 5).

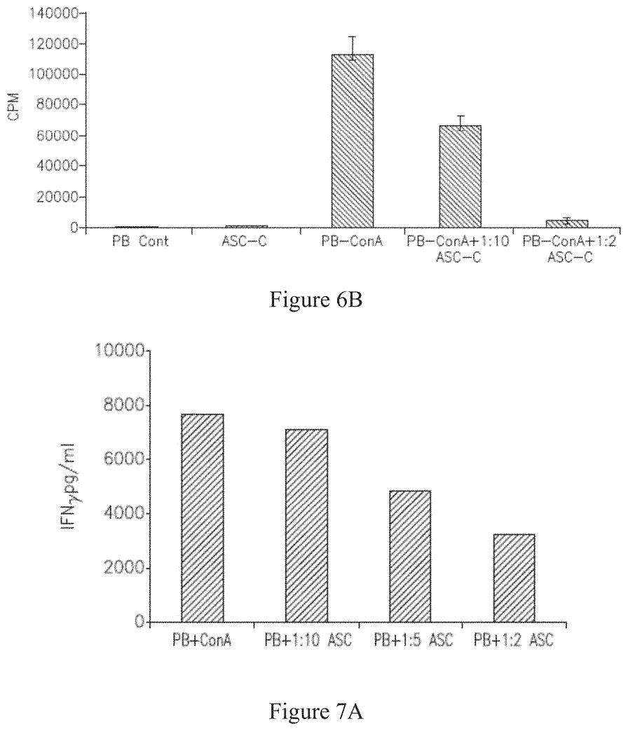

MLR performed with 2 different donors also showed that the ASC both escaped allorecognition (FIG. 6A) and reduced lymphocyte proliferation, as measured by thymidine incorporation, following mitogenic stimuli, such as Concanavalin A (Con A) (FIG. 6B) and Phytohemagglutinin (PHA; typically at least 25% inhibition relative to PHA alone), and non-specific stimulation by anti-CD3 and anti-CD28. The reduction in lymphocyte proliferation was dose dependent with the number of ASC.

Next, PBMC were stimulated by PHA using the Transwell® method (which prevents cell-to-cell contact but enables the diffusion of cytokines between the two compartments). The inhibition of proliferation was maintained even in this assay, showing that cell-to-cell contact was not necessary for the inhibition.

### Example 7. Adherent Stromal Cells Alter Cytokine Secretion by PBMC and Stimulate Endothelial Cell Proliferation

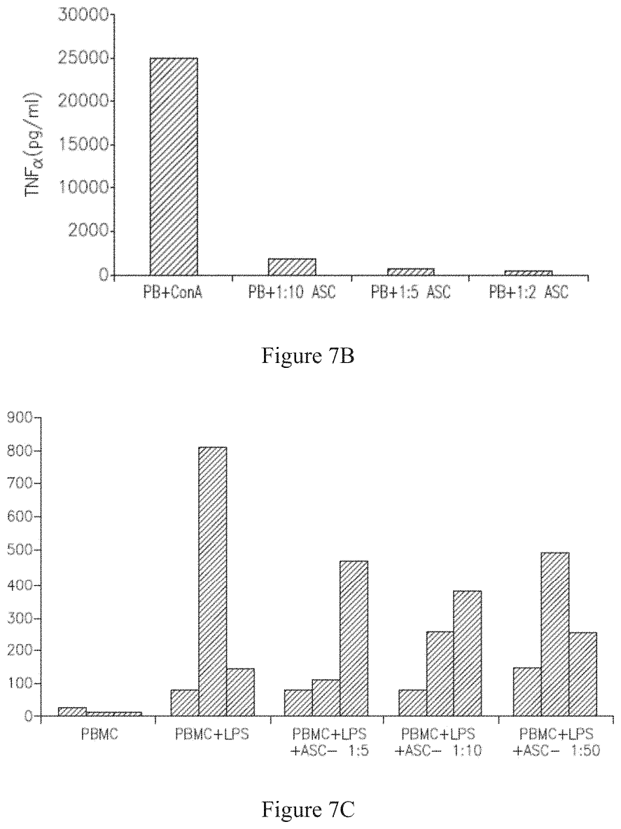

Additional co-culture studies were performed to test the effect of ASC on secretion of cytokines by lymphocytes. Culturing of PB-derived mononuclear cells (PBMC) with ASC slightly reduced IFN-gamma secretion and dramatically reduced TNF-alpha secretion by the PBMC, even when only low amounts of ASC were present (FIGS. 7A-B). Under conditions of LPS stimulation, the ASC increased secretion of IL-10 by PBMC, while decreasing their secretion of TNF-alpha, in a dose-dependent manner (FIG. 7C).

### Protocol—Endothelial Cell Proliferation (ECP) Assay:

ASC were prepared as described in Example 1, harvested by vibration, as described in PCT International Application Publ. No. WO 2012/140519, and were cryopreserved. 1×10⁶ thawed ASC were seeded in 2 ml DMEM medium. After 24 hours (hr), the medium was replaced with EBM-2 medium (Lonza Group Ltd, Basel, Switzerland), and cells were incubated under hypoxic conditions (1% O₂) for an additional 24 hr, after which the conditioned media was collected. In parallel, 750 human umbilical cord endothelial cells (HUVEC) were seeded, incubated for 24 hr, and then incubated with the conditioned media, for 4 days under normoxic conditions at 37° C. After removal of the conditioned medium, the proliferation of the HUVEC cells was assayed using the AlamarBlue® fluorescent assay. Results are presented as the percent ECP (% ECP) observed after PHA stimulation in the absence of ASC (arbitrarily set at 100%/).

### Results

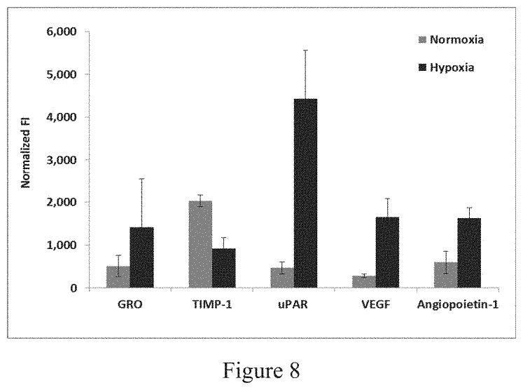

ASC cultured under normoxic or hypoxic conditions were tested for protein secretion, using Cytokine (Human) Antibody Array C Series 4000 (RayBio). Secretion of several pro-angiogenic factors was up-regulated under hypoxic conditions, as shown in FIG. 8.

In additional experiments, various batches of ASC were co-incubated with HUVEC cells to test their effect on ECP. Stimulation of ECP was observed, typically at least 135% of the ECP observed in the absence of ASC.

### Example 8: Treatment of ASC with Pro-Inflammatory Cytokines During 3D Culturing

Methods

General experimental protocol. ASC were obtained from the placenta and cultured under 2D conditions, then under 3D conditions, and were then harvested, all as described in Example 1, with the following deviation: One day before the end of the 3D culture (typically on day 5 or 6), the medium was replaced with DMEM, with or without the addition of 10 nanograms/milliliter (ng/ml) Tumor Necrosis Factor alpha (TNF-alpha), 10 ng/ml Interferon-Gamma (IFN-g), and/or 10% FBS (see Table 5), and the bioreactor was incubated in batch mode (or, in selected experiments, in perfusion mode) for an additional day. Levels of secreted cytokines were measured in the bioreactor medium, using the RayBio® Human Cytokine Array kit.

Hypoxic incubation. 1×10⁶ thawed ASC were seeded in 2 ml DMEM medium. After 24 hours (hr), the medium was replaced with EBM-2 medium (Lonza Group Ltd, Basel, Switzerland), and cells were incubated under hypoxic conditions (1% 02) for an additional 24 hr, after which the conditioned media was collected.

TABLE 5
Incubation conditions that were tested.

| Designation | Cytokines | FBS |
|---|---|---|
| 1 | None | NO |
| 2 | None | YES |
| 3 | TNF | NO |
| 4 | TNF | YES |
| 5 | TNF + IFN | NO |
| 6 | TNF + IFN | YES |

In other experiments, levels of secreted cytokines were measured in the conditioned medium (CM) from a hypoxic incubation, as described above.

Quantitative detection of secreted proteins: IL-6 was quantitatively measured using the human IL-6 immunoassay Quantikine® ELISA kit (R&D Systems). VEGF was quantitatively measured using the Human VEGF immunoassay Quantikine® kit (R&D Systems).

Results

In a series of experiments testing various conditions side-by-side, adherent stromal cells (ASC) were incubated in a bioreactor as described in the previous Examples. On the last day of the bioreactor incubation, the medium was replaced by medium containing or lacking added TNF-alpha and/or IFN-gamma, in the presence or absence of FBS. VEGF and IL-6 secretion were measured by ELISA. Inclusion of TNF-alpha significantly increased secretion of VEGF, whether or not IFN-gamma was present (Table 6).

TABLE6
Secretion of VEGF (picograms/ml [pg/ml])
by ASC under various conditions.

| | | | VEGF in | VEGF in bioreactor |
|---|---|---|---|---|
| Expt. # | Cytokines | FBS | CM/RPD* | medium/RPD* |
| 1 | TNF + IFN | NO | 619/3 | 195/3 |
| | None | NO | 274/7 | 65/0 |
| 2 | TNF + IFN | NO | 7540/1 | 151/3 |
| | None | NO | 3266/4 | 140/3 |
| 3 | TNF + IFN | YES | 371/3 | 1749/2 |
| | INI | YES | 370/10 | 1128/5 |
| 4 | TNF + IFN | YES | NT (not tested) | 373/2 |
| | TNF | YES | NT | 348/8 |
| 5 | TNF + IFN | NO | 732 ± 20** | (not performed) |
| | None | NO | 650 ± 46** | (not performed) |

*In this table and throughout the document, RPD refers to the percentage difference between duplicate samples in the ELISA.
**Indicated number is the standard deviation.

In the same experiment, inclusion of TNF-alpha significantly increased IL-6 secretion, which was further increased by IFN-gamma, as shown in Table 7.

TABLE 7
Secretion of IL-6 (picograms/rnl [pg/ml])
by ASC under various conditions.

| Expt. | Cytokines | FBS | IL-6 in CM | RPD |
|---|---|---|---|---|
| 1 | TNF + IFN | NO | 77 | 2 |
| | None | NO | 10 | 2 |
| 2 | TNF + IFN | NO | 509* | 1 |
| | None | NO | 40 | 4 |
| 3 | TNF + IFN | YES | 380 | 0 |
| | TNF | YES | 92 | |

*above calibration curve.

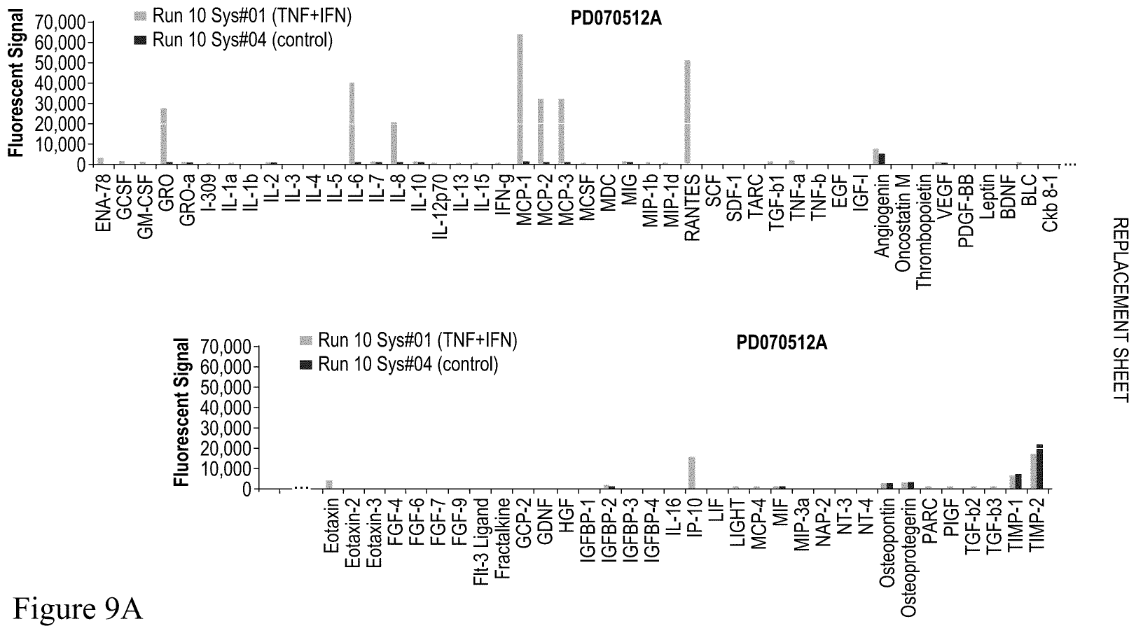

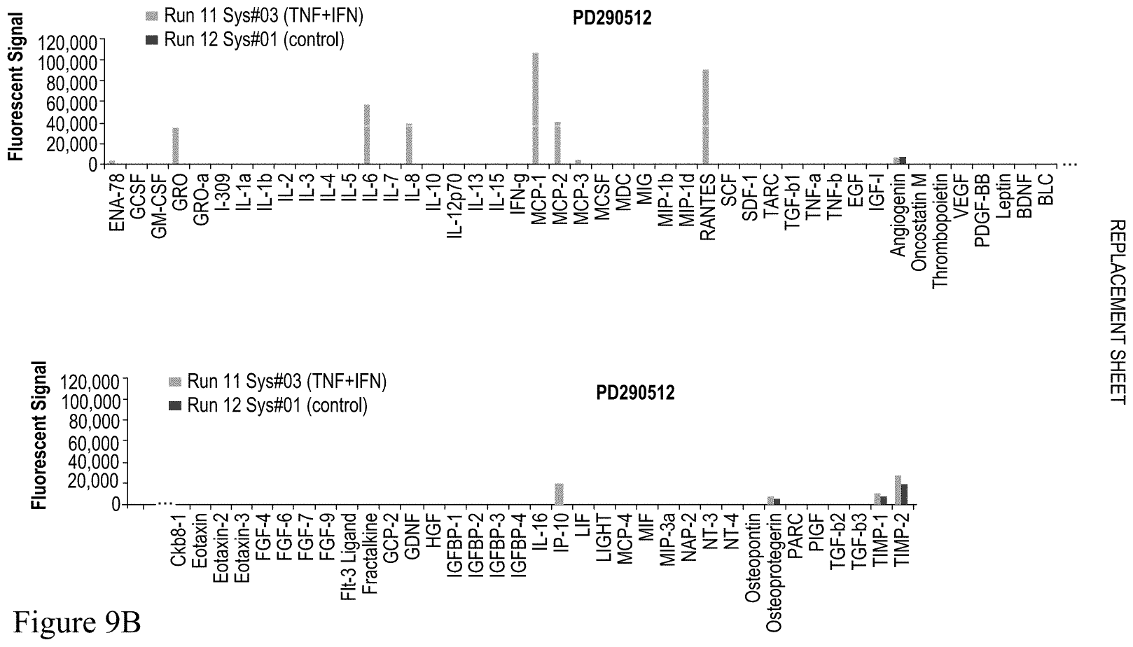

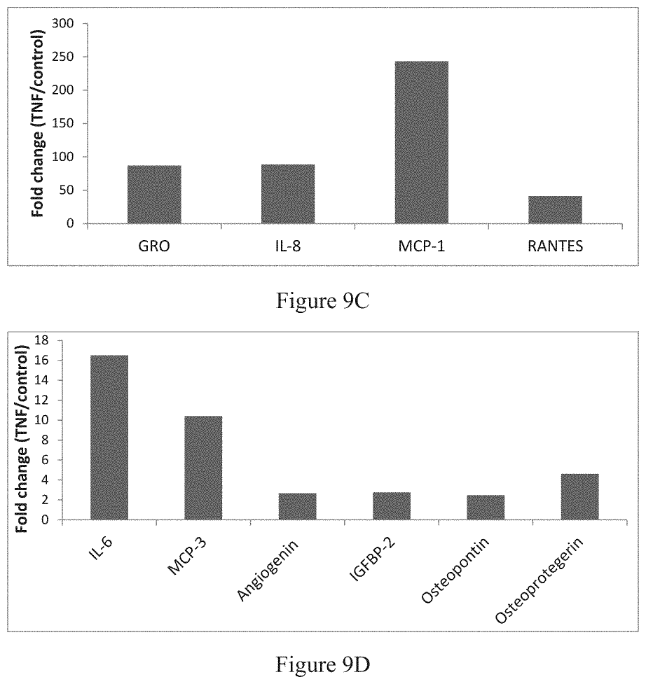

Expression of a panel of factors in the bioreactor media of Experiments 1-2 (see Tables 6-7), all performed in the absence of serum, was measured by a fluorescence-based cytokine array assay, revealing the increased expression of several factors, including GRO, IL-6, IL-8, MCP-1, MCP-2, MCP-3, RANTES, and IP-10 (Experiments 1-2 are shown in FIGS. 9A-B, respectively). In another experiment, TNF-alpha alone was compared to medium without cytokines (also in the absence of serum), showing increased expression of GRO, IL-8, MCP-1, RANTES, and, to a lesser extent, IL-6, MCP-3, Angiogenin, Insulin-like Growth Factor Binding Protein-2 (IGFBP-2), Osteopontin, and Osteoprotegerin (FIGS. 9C-D).

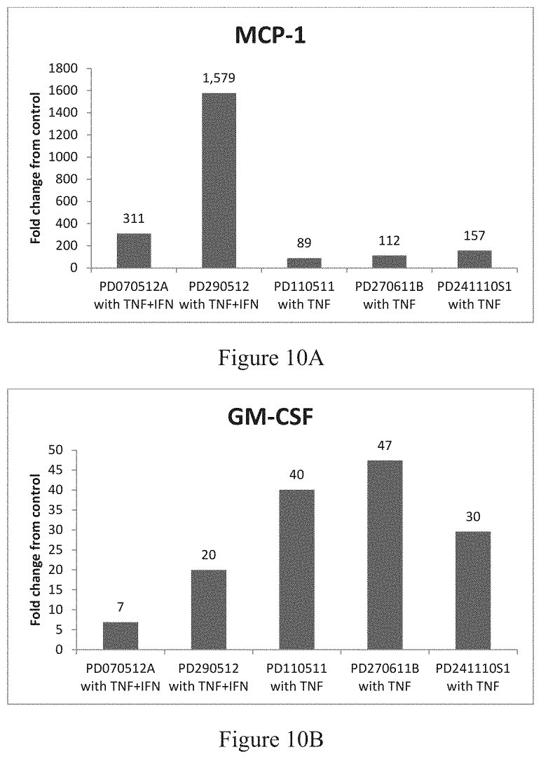

Increased expression of MCP-1 and GM-CSF in the bioreactor media was verified by quantitative ELISA in several experiments, all performed in the absence of serum. The results showed that TNF-alpha+IFN-gamma was superior to TNF-alpha alone for MCP-1 induction (FIG. 10A), while TNF-alpha alone appeared to be slightly superior for GM-CSF induction (FIG. 10B). The cytokine concentrations and fold-changes relative to control medium (containing no cytokines) from the TNF-alpha+IFN-gamma trial are shown in Table 8 below.

TABLE 8
MCP-1 and GM-CSF concentrations in bioreactor medium.

| | | MCP-1 (pg/ml) | | GM-CSF (pg/ml) | |
|---|---|---|---|---|---|
| Expt. No. | Conditions | (fold-increase) | | (fold-increase) | |
| 1 | TNF + IFN | 6365.4 | (311) | 6.32 | (6.9) |
| | None | 20.5 | | 0.91 | |
| 2 | TNF + IFN | 9063.7 | (1579) | 13.09 | (20.0) |

| | | |
|---|---|---|
| None | 5.8 | 0.65 |

### Example 9: The Effect of Serum on Pro-Inflammatory Cytokine Treatment of ASC During 3D Culturing

The next experiment examined the effect of FBS on induction of the aforementioned panel of factors by TNF-alpha+IFN-gamma or TNF-alpha alone. A similar set of major proteins was induced in the presence or absence of FBS. In the case of TNF-alpha alone, IL-6 appeared to be induced much more strongly in the presence of FBS than in its absence.

### Example 10: Quantitative RANTES ELISA on ASC

ASC were incubated with 10 ng/ml TNF-alpha, alone or in combination with 10 ng/ml IFN-gamma, as described for Example 9. The cells were cryopreserved, then thawed, and then 5×10⁵ cells were seeded in DMEM supplemented with 10% FBS and incubated under standard conditions. After 24 hours, the medium was replaced with 1-ml serum-free medium, and the cells were incubated another 24 hours under normoxic conditions. The medium was removed and assayed for RANTES secretion by ELISA, using the Quantikine® ELISA Human CCL5/RANTES kit (R&D Systems). The TNF-alpha+IFN-gamma-treated cells had sharply upregulated RANTES secretion compared to the other groups (Table 9).

TABLE 9
RANTES concentrations in culture medium.

| | | RANTES | Standard |
|---|---|---|---|
| Expt. No. | Conditions | conc. | dev. |
| 5 | No cytokines, no serum | 0 | 0 |
| 7 | No cytokines, serum. | 2 | 1 |
| 8 | No cytokines, serum | 0 | 0 |
| 5 | TNF-alpha, no serum | 76 | 2 |
| 7 | TNF-alpha, serum. | 591 | 20 |
| 8 | IFN-gannna + TNF-alpha + serum. | 3232* | 83 |

*Out of calibration curve.

### Example 11: Treatment of Viral-Induced Pneumonia and Complications with Placental ASC

Methods

Data Analysis

All data analyses were conducted in version 3.6.1 of R (R Core Team, 2019). Using the lmerTest package, baseline adjusted repeated measures linear models were fit with restricted maximum likelihood (REML), and t-tests were computed using Satterthwaite's method. Time points in which only a single subject contributed data were omitted before running the repeated measures models.

Patient Characteristics

Eight patients (7 males and 1 female) were treated. All patients were confirmed for SARS-CoV-2 infection by real-time reverse transcriptase polymerase chain reaction (RT-PCR). The median age of the patients was 55 years (range 22-79 years). ⅝ patients were at higher risk for severe illness from COVID-19 due to underlying medical conditions. The most common comorbidities were hypertension (4 patients) and diabetes (4 patients), with 3 patients suffering from both. 7 patients had BMI above 25. None of the patients were active smokers. Prior to ASC treatment, all patients had received hydroxychloroquine, 3 patients received lopinavir/ritonavir, and 2 patients received remdesivir. In addition, 3 patients had received IL-6 inhibitors, and 4 patients had received steroids. Two patients needed Extracorporeal Membrane Oxygenation (ECMO). The first, patient #6, needed resuscitation due to a massive pulmonary embolism, occurring 4 days after ASC treatment, and was placed on ECMO until full recovery. The second, #4, was electively placed on ECMO due to severe non-resolving ARDS and hypoxia. The average length of hospital stay was 41 days for the six patients being discharged by follow-up end. ⅝ patients were intubated for at least 5 days before ASC treatment, with 1 patient (#8) being intubated for 22 days prior to ASC treatment.

Results

Eight 2019-nCoV-infected patients, all in Intensive Care Units (ICU) on invasive mechanical ventilation and suffering from Acute Respiratory Distress Syndrome (ARDS), were enrolled in a compassionate use program over a 9-day period. Patients were administered 300 million maternal placental ASC in a mixture containing 10% DMSO, 5% human serum albumin and Plasma-Lyte®, via 15 IM injections (1 mL each). ⅜ received an additional treatment of 300 million cells 8 or 11 days later, according to the physician's discretion.

COVID-19-infected patients that require invasive mechanical ventilation are considered at high risk for mortality (Bhatraju P K et al.). Despite that, the patients studied exhibited a 100% survival rate at the first assessment, which was at least 1 week after treatment for 6/7 of the patients and 2 days after treatment for the 7^{th}. Furthermore, 4 patients exhibited multi-organ failure prior to treatment; 2/4 (50%) exhibited clinical recovery in addition to the respiratory improvement.

No adverse events attributed to ASC treatment were reported.

Improvement in CRP Levels

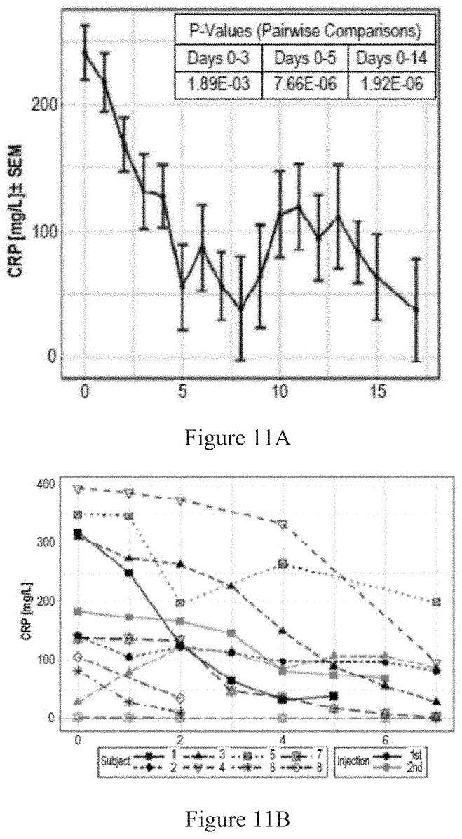

By day 3 post injection, mean CRP had fallen 45% (240.3 mg/L to 131.3 mg/L, p=1.9×10³) and by day 5, 77% (to 56.0 mg/L, p=7.7×10⁻⁶) (FIG. 11A). The drop in blood CRP following injection of ASC either on day 0 (data from all subjects is shown) or, if the subject had a second injection, on either day 8 or 11 (day 11 for subject #1 or day 8 for subjects #3 and #7) is shown in FIG. 11B. These data generally show that the higher the level of blood CRP, the more dramatic the reduction following treatment.

Improvement in Respiratory Parameters

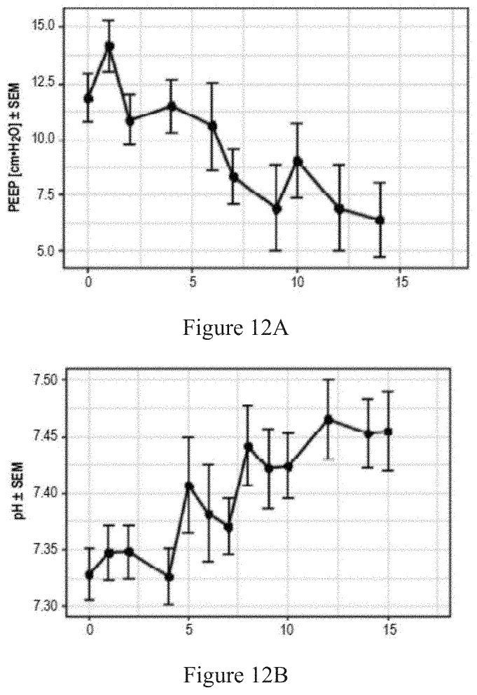

PaO₂/FiO₂ increased in ⅝ patients 24 h post treatment, with a similar effect 48 h post treatment (Table 10). A decrease in PEEP (Positive End Expiratory Pressure) and increase in pH were both statistically significant between days 0-14 (p=3.2×10³ and p=7.2×10⁻⁴, respectively; FIGS. 12A-B).

TABLE 10
P/F ratio of patients before and after treatment.

| Patent | Before | 24 hr Post | 48 hr Post |
|---|---|---|---|
| No. | Treatment | Treatment | Treatment |
| 1 | 160 | 229 | 170 |
| 2 | 140 | 172.5 | 177.5 |
| 3 | 143 | 151 | 217 |
| 4 | 149 | 107 | NA |
| 5 | 106 | 145 | NA |
| 6 | 173 | 205 | NA |
| 7 | 172 | 93 | 95 |
| 8 | 342.5 | NA | 425 |

Most of the patients (6 out of 8) required vasopressor treatments prior to enrollment. In 3 patients, vasopressor doses could be decreased following ASC treatments and/or discontinued as early as 3 days post treatment (patient #3).

Changes in Chest Radiographs

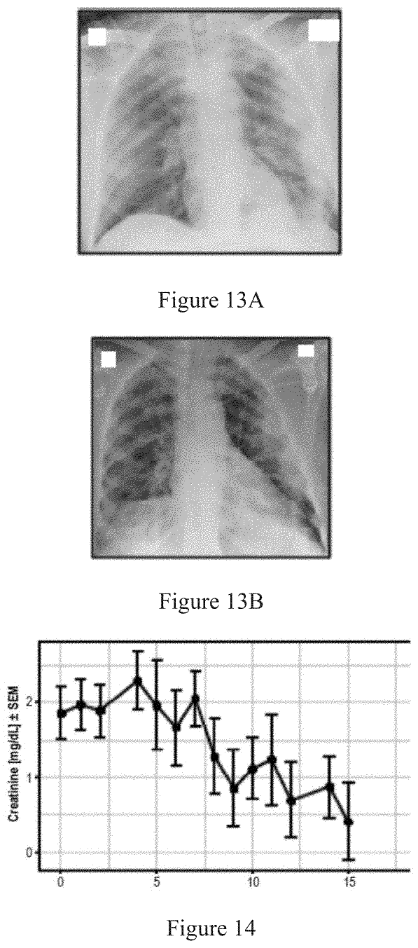

Chest radiographs were obtained from 6 patients. In patients #1 and #3, the radiographs demonstrated some resolution and improvement in the interstitial opacities. FIG. 13 shows image from patient #3. Some improvement was also seen in patients #5, #6, and #8. No improvement was seen in patient #4. (Data prior to ASC treatment was not available for patients #2 and #7).

Improvement in Kidney Function

A decrease in creatinine, indicative of acute kidney injury, was statistically significant between days 0-14 (p=3.2×10²; FIG. 14), representing a 53% drop (from 1.875 to 0.883 mg/dL).

28-Day Follow Up Data

After a 28-day follow-up period, ⅞ (87.5%) of the subjects were still alive, 6/8 (75%) had been weaned off mechanical ventilation, and ⅝ (62.5%) had been discharged alive from the hospital. By contrast, mortality rates of ARDS COVID-19 patients who require mechanical ventilation ranged (around the time of this study) between 24.5% to 94% in different studies (Richardson S et al. and Gibson P G et al.)

### Example 12: Use of ASC in Treating HIV-1 Infection

ASC are tested in a culture model of Human Immunodeficiency Virus 1 (HIV-1). Non-limiting examples are cell lines, for example J-Lat A1 cells, and primary cells, for example primary CD4 T cells and dendritic cells. The experiments may be carried out, for example, as described in Jiang G et al or Nasr N et al, and the references cited therein. HIV-1 replication can be quantified by detection of an HIV-1 antigen (for example HIV-1 gag p24 antigen detection [see, for example, Yang H et al, 2013, and the references cited therein]), or by fluorescence-based systems, such as PCR (see, for example, Vermeire J et al, 2012, and the references cited therein). In still other experiments, conditioned medium or exosomes are added instead of the ASC. In each of the above cases, the virus or viral construct can be modified to express a reporter gene, for example luciferase, which is used to quantify viral replication. In other experiments, HIV-1 infection is assessed in an animal model, for example primates such as rhesus macaque or sooty mangabey, or rodents, such as T-cell humanized mice, for example expressing human CD4, CCR5 and/or cyclin T1. The models may utilize HIV-1 or similar viruses, for example attenuated or wild-type HIV-1, simian/human immunodeficiency virus (SHIV), or simian immunodeficiency virus (SIV). Experiments are carried out, for example, as described in Zhang W J et al, Roederer M et al, Rotger M et al, or Chang J J et al, and the references cited therein. In still other experiments, human subjects with HIV-1 are administered compositions comprising ASC, and viral infection levels, viral persistence, secondary infections, and/or symptoms are assessed. Inhibition of replication and/or amelioration of infection is evidence of therapeutic efficacy.

### Example 13: Use of ASC in Treating HCV Infection

ASC are tested in a culture model of hepatitis C virus (HCV), non-limiting examples of which are liver slices, for example as described in Lagaye S et al and the references cited therein; and helper-dependent culture models or other culture models, for example as described in Zhou X1 et al, Steinmann E et al, or Yang D et al, and the references cited therein. In some experiments, target cells, for example hepatoma cells, are engineered to carry a subgenomic HCV construct (see, for example, Wakita T et al, 2005, and the references cited therein), or a subgenomic HCV construct is introduced to target cells, and the described ASC are added to the culture. In still other experiments, conditioned medium or exosomes are added instead of the ASC. In each of the above cases, the virus or viral construct can be modified to express a reporter gene, which is used to quantify viral replication. In other experiments, HCV infection is studied in an animal model, utilizing HCV or similar viruses, for example attenuated or wild-type HCV, for example as described in Tesfaye A et al. and the references cited therein. In still other experiments, human subjects with HCV are administered compositions comprising ASC, and viral infection levels, viral persistence, and/or symptoms are assessed. Inhibition of replication and/or amelioration of infection is evidence of therapeutic efficacy.

### Example 14: Use of ASC in Treating HBV Infection

ASC are tested in a culture model of hepatitis B virus (HBV), for example as described in Ahn S el al., 2014 or Yang D et al., and the references cited therein. In some experiments, target cells, for example hepatoma cells, are engineered to carry the HBV genome or portion thereof, and the effect of addition of ASC to the cultures is assessed. Viral replication can be measured by detecting HBV RNA levels, levels of pregenomic RNA, viral DNA, and levels or virus particles, virus-like particles, virus progeny, or other virus markers (for example HBsAg) in the cell culture medium. In still other experiments, conditioned medium or exosomes are added instead of the ASC. In each of the above cases, the virus or viral construct can be modified to express a reporter gene, which is used to quantify viral replication. In other experiments, HBV infection is studied in an animal model, utilizing HBV or similar viruses, for example attenuated or wild-type HBV, for example as described in Tesfaye A et al, and the references cited therein. In still other experiments, human subjects with HBV are administered compositions comprising ASC, and viral infection levels, viral persistence, and/or symptoms are assessed. Inhibition of replication and/or amelioration of infection is evidence of therapeutic efficacy.

### Example 15: Use of ASC in Treating HSV Infection

ASC are tested in a culture model of herpes simplex virus (HSV), for example HSV-1 or HSV-2. Non-limiting examples of models are described in Kao Y S et al and Zabihollahi R et al, and the references cited therein. In some experiments, target cells, for example epithelial cells or neuronal cells, are infected with HSV or a modified form thereof, and the effect of addition of ASC to the cultures is assessed. In still other experiments, conditioned medium or exosomes are added instead of the ASC. In each of the above cases, the virus or viral construct can be modified to express a reporter gene, which is used to quantify viral replication. In other experiments, HSV infection is studied in an animal model. For example, viral titer in particular organ(s) can be assessed. In some experiments, viral titer in the liver is measured, for example as described in Sorensen L N et al and the references cited therein; or viral infection in the cornea is measured, for example as described in Terrell et al and the references cited therein. In still other experiments, human subjects with HSV are administered compositions comprising ASC, and viral infection levels, viral persistence, and/or symptoms are assessed. Inhibition of replication and/or amelioration of infection is evidence of therapeutic efficacy.

### Example 16: Use of ASC in Treating Dengue Virus Infection

ASC are tested in a culture model of Dengue virus, non-limiting examples of which utilize kidney cells, for example BHK-21 cells or Vero cells, as described in Kato F et al 2014 and the references cited therein, hepatoma cells, for example Huh7 cells as described in Miller S et al 2006 and the references cited therein. In some experiments, target cells are infected with or engineered to carry Dengue virus or a subgenomic construct thereof, and ASC are added to the culture. Viral replication can be measured by detecting virus progeny, for example as described in Carter J R et al 2013. In still other experiments, conditioned medium or exosomes are added instead of the ASC. In each of the above cases, the virus or viral construct can be modified to express a reporter gene, which is used to quantify viral replication. In other experiments, Dengue infection is studied in an animal model, for example rodents deficient in the interferon-α/β and interferon-γ receptor or non-human primates such as rhesus macaques (Macaca mulatta), as described in as described in Byrd C M et al., 2013, Whitehorn J et al., 2014, and the references cited therein. In other experiments, Dengue infection is studied in humans, for example as described in Mammen M P el al., 2014 and the references cited therein, and viral infection levels, viral persistence, and/or symptoms, for example fever, are assessed. Inhibition of replication and/or amelioration of infection is evidence of therapeutic efficacy.

### Example 17: Use of ASC in Treating Ebola Virus Infection

ASC are tested in a culture model of Ebola virus, for example cells carlying the Ebola virus genome, a fraction thereof, or a modified version thereof, examples of which are described in Groseth A el al., 2005, Groseth A el al., 2012, Neumann G el al., 2002, and the references cited therein. In still other experiments, conditioned medium or exosomes are added instead of the ASC. In each of the above cases, the virus or viral construct can be modified to express a reporter gene, for example chloramphenicol acetyltransferase (CAT) or green fluorescent protein (GFP), which is used to quantify viral replication. In other experiments, Ebola infection is studied in an animal model, for example mouse-adapted Zaire ebolavirus, as described in Chen H el al., 2013 and the references cited therein; and non-human primates, for example cynomolgus macaque (Macaca fascicularis) and rhesus macaque, as described in Rubins K H el al., 2007, Smith L M 2013, and the references cited therein. Viral titer, serum cytokine levels, and expression in PBMC of cytokines, chemokines, death proteins such as TRAIL, and/or fibrin-related genes can be used to monitor viral infection. In still other experiments, human subjects with Ebola are administered compositions comprising ASC, and viral infection levels, and/or symptoms are assessed. Inhibition of replication and/or amelioration of infection is evidence of therapeutic efficacy.

### Example 18: Use of ASC for Treating Hemorrhagic Fever

ASC are tested in an animal model of Hemorrhagic Fever (HF) and/or cytokine storm. Classically, HF is caused by viruses classified as filoviruses (for example Ebola and Marburg virus), flaviviruses (for example yellow fever virus and Dengue virus), arenaviruses (for example Lassa virus), and bunyaviruses (for example Crimean-Congo HFV and Rift Valley virus). In other experiments, other viruses such as influenza are examined as model systems. In still other experiments, virus-like particles from Ebola or other immunogenic viruses are used as model systems. In some experiments, serum levels of inflammatory cytokines, for example interleukin [IL]-1β, IL-6, and TNF-α, and/or expression in PBMC of cytokines, chemokines, death proteins such as TRAIL, and/or fibrin-related genes are used to monitor the course of HF. In other experiments, indicators of hypercytokinemia, systemic inflammatory response syndrome (SIRS), or dysregulated coagulation are measured, for example as described in Bray M et al, 2001, Geisbert T W el al., 2003, Yen J Y et al., 2011, and references cited therein. In other experiments, indicators of septic shock are measured, for example as described in Bray M et al, 2003, and references cited therein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES (ADDITIONAL REFERENCES MAY BE CITED IN TEXT)

Ahn S et al., An in vitro liver model consisting of endothelial vascular networks surrounded by human hepatoma cell lines allows for improved hepatitis B virus replication. J Biosci Bioeng. 2014 July; 118(1):107-11.
Bhatraju P K et al., Covid-19 in Critically Ill Patients in the Seattle Region—Case Series. N Engl J Med. 2020 Mar. 30. doi: 10.1056/NEJMoa2004500. [Epub ahead of print]
Bray M el al., Haematological, biochemical and coagulation changes in mice, guinea-pigs and monkeys infected with a mouse-adapted variant of Ebola Zaire virus. J Comp Pathol. 2001 November; 125(4):243-53.
Byrd C M et al, A novel inhibitor of dengue virus replication that targets the capsid protein. Antimicrob Agents Chemother. 2013 January; 57(1):15-25.
Carter J R et al, A novel dengue virus detection method that couples DNAzyme and gold nanoparticle approaches. Virol J. 2013 Jun. 28; 10:201.
Chang J J et al. Immune activation and the role of TLRs and TLR agonists in the pathogenesis of HIV-1 infection in the humanized mouse model. J Infect Dis. 2013 November; 208 Suppl 2:S145-9.
Chase L G et al., Development and Characterization of a Clinically Compliant Xeno-Free Culture Medium in Good Manufacturing Practice for Human Multipotent Mesenchymal Stem Cells. Stem Cells Transl Med. 2012 October; 1(10): 750-758.
Chen H et al., Myxomavirus-derived serpin prolongs survival and reduces inflammation and hemorrhage in an unrelated lethal mouse viral infection. Antimicrob Agents Chemother. 2013 September; 57(9):4114-27.
Dominici et al., Minimal criteria for defining multipotent mesenchymal stromal cells. The International Society for Cellular Therapy position statement. Cytotherapy. 2006; 8(4):315-7.
Geisbert T W et al., Mechanisms underlying coagulation abnormalities in ebola hemorrhagic fever: overexpression of tissue factor in primate monocytes/macrophages is a key event. J Infect Dis. 2003 Dec. 1; 188(11):1618-29.
Gibson P G et al., COVID-19 ARDS: clinical features and differences to “usual” pre-COVID ARDS. The Medical Journal of Australia. 2020. [Preprint, 24 Apr. 2020].
Groseth A et al, RNA polymerase I-driven minigenome system for Ebola viruses. J Virol. 2005 April; 79(7):4425-33.
Groseth A et al, The Ebola virus glycoprotein contributes to but is not sufficient for virulence in vivo. PLoS Pathog. 2012; 8(8):e1002847.
Jiang G et al. Reactivation of HIV latency by a newly modified Ingenol derivative via protein kinase Cδ-NF-κB signaling. AIDS. 2014 Jul. 17; 28(11):1555-66.
Kao Y S et al. Persistent infection of a lymphoma cell line by herpes simplex virus. Am J Hematol. 1999 October; 62(2):93-8.
Kato F et al, Development of a novel dengue-1 virus replicon system expressing secretory Gaussia luciferase for analysis of viral replication and discovery of antiviral drugs. Jpn J Infect Dis. 2014; 67(3):209-12.
Kinzebach S and Bieback K. Expansion of Mesenchymal Stem/Stromal cells under xenogenic-free culture conditions. Adv Biochem Eng Biotechnol. 2013; 129:33-57.
Lagaye S et al., Efficient replication of primary or culture hepatitis C virus isolates in human liver slices: a relevant ex vivo model of liver infection. Hepatology. 2012 September; 56(3):861-72.
Mammen M P et al., Evaluation of dengue virus strains for human challenge studies. Vaccine. 2014 Mar. 14; 32(13):1488-94.
Miller S et al., Subcellular localization and membrane topology of the Dengue virus type 2 Non-structural protein 4B. J Biol Chem. 2006 Mar. 31; 281(13):8854-63.
Mimura et al, Growth factor-defined culture medium for human mesenchymal stem cells. Int. J. Dev. Biol. 55: 181-187 (2011).
Monaca E et al, Assessment of hemostaseologic alterations induced by hyperbaric oxygen therapy using point-of-care analyzers. Undersea Hyperb Med. 2014 January-February; 41(1):17-26.
Nasr N et al., HIV infection of dendritic cells. Methods Mol Biol. 2014; 1087:221-32.
Neumann G et al., Reverse genetics demonstrates that proteolytic processing of the Ebola virus glycoprotein is not essential for replication in cell culture. J Virol. 2002 January; 76(1):406-10.
Richardson S el al., Presenting Characteristics, Comorbidities, and Outcomes Among 5700 Patients Hospitalized With COVID-19 in the New York City Area. JAMA 2020. doi:10.1001/jama.2020.6775
Robinson J, Halliwell J A, Hayhurst J H, Flicek P, Parham P, Marsh S G E: The IPD and IMGT/HLA database: allele variant databases. Nucleic Acids Research. 2015 43:D423-431.
Roederer M et al., Immunological and virological mechanisms of vaccine-mediated protection against SIV and HIV. Nature. 2014 Jan. 23; 505(7484):502-8.
Rotger M et al., Comparative transcriptomics of extreme phenotypes of human HIV-1 infection and SIV infection in sooty mangabey and rhesus macaque. J Clin Invest. 2011 June; 121(6):2391-400.
Rubins K H et al., The temporal program of peripheral blood gene expression in the response of nonhuman primates to Ebola hemorrhagic fever. Genome Biol. 2007; 8(8):R174.
Smith L M et al., Interferon-p therapy prolongs survival in rhesus macaque models of Ebola and Marburg hemorrhagic fever. J Infect Dis. 2013 Jul. 15; 208(2):310-8.
Sørensen L N et al., Blocking C C chemokine receptor (CCR) 1 and CCR5 during herpes simplex virus type 2 infection in vivo impairs host defence and perturbs the cytokine response. Scand J Immunol. 2004 March; 59(3):321-33.
Steinmann E et al., Cell culture systems for hepatitis C virus. Curr Top Microbiol Immunol. 2013; 369:17-48.
Terrell S L et al., Roles of conserved residues within the pre-NH2-terminal domain of herpes simplex virus 1 DNA polymerase in replication and latency in mice. J Gen Virol. 2014 April; 95(Pt 4):940-7.
Tesfaye A et al., Chimeric mouse model for the infection of hepatitis B and C viruses. PLoS One. 2013 Oct. 14; 8(10):e77298.
Vermeire J et al., Quantification of reverse transcriptase activity by real-time PCR as a fast and accurate method for titration of HIV, lenti- and retroviral vectors. PLoS One. 2012; 7(12):e50859.
Wakita T et al., Production of infectious hepatitis C virus in tissue culture from a cloned viral genome. Nat Med. 2005 July; 11(7):791-6.
Whitehorn J, Dengue Therapeutics, Chemoprophylaxis, and Allied Tools: State of the Art and Future Directions. PLoS Negl Trop Dis. 2014 Aug. 28; 8(8):e3025.
Yang D et al., Complete replication of hepatitis B virus and hepatitis C virus in a newly developed hepatoma cell line. Proc Natl Acad Sci USA. 2014 Apr. 1; 111(13):E1264-73.
Yang H et al., Improved quantification of HIV-1-infected CD4+ T cells using an optimised method of intracellular HIV-1 gag p24 antigen detection. J Immunol Methods. 2013 May 31:391(1-2):174-8.
Yen J Y et al., Therapeutics of Ebola hemorrhagic fever: whole-genome transcriptional analysis of successful disease mitigation. J Infect Dis. 2011 November; 204 Suppl 3:S1043-52.
Zabihollahi R et al., Inhibition of HIV and HSV infection by vaginal lactobacilli in vitro and in vivo. Daru. 2012 Oct. 15; 20(1):53.
Zhang W J et al., Associated changes in the transcription levels of IL-17A and tight junction-associated genes in the duodenal mucosa of rhesus macaques repeatedly exposed to simian/human immunodeficiency virus. Exp Mol Pathol. 2014 Jul. 14.
Zhou X I et al., A novel helper-dependent adenovirus-based cell culture model for Hepatitis C virus replication and production. Virol J. 2013 Aug. 30; 10:273.

What is claimed is:

1. A method for treating or ameliorating a virus infection, comprising administering a composition that comprises a cultured placental adherent stromal cell (ASC), thereby treating or ameliorating a virus infection.

2. A method for treating, preventing, or ameliorating a complication of a virus infection, comprising administering a composition that comprises a cultured placental adherent stromal cell (ASC), thereby treating, preventing, or ameliorating a complication of a virus infection.

3. The method of claim 1, where said virus is selected from HIV-1, HCV, HBV, HSV-1, HSV-2, Dengue virus, Marburg virus, Ebola virus, yellow fever virus, Lassa virus, Crimean-Congo HFV, and Rift Valley virus.

4. The method of claim 1, where said composition is an injected composition.

5. The method of claim 1, wherein said placental ASC have been incubated on a 2D substrate.

6. The method of claim 1, wherein said placental ASC have been incubated on a 3D substrate.

7. The method of claim 6, wherein said placental ASC have been incubated on a 2D substrate, prior to incubating on a 3D substrate.

8. The method of claim 7, wherein said 3D culture substrate comprises a fibrous matrix, comprising a synthetic adherent material, where said synthetic adherent material is selected from the group consisting of a polyester, a polypropylene, a polyalkylene, a polyfluorochloroethylene, a polyvinyl chloride, a polystyrene, and a polysulfone.

9. The method of claim 8, wherein said 3D culture apparatus is in form of microcarriers, wherein said microcarriers are disposed in a bioreactor.

10. The method of claim 1, wherein said placental ASC is allogeneic to said subject.

11. The method of claim 1, wherein the composition is intramuscularly injected.

12. The method of claim 1, comprising 100-600 million of said placental ASC, for an adult subject.

13. The method of claim 1, wherein said composition comprises:
a. a first pharmaceutical composition, comprising allogeneic placental ASC from a first donor; and
b. a second pharmaceutical composition, comprising allogeneic placental ASC from a second donor, wherein said second donor differs from said first donor in at least one allele group of human leukocyte antigen (HLA)-A or human leukocyte antigen (HLA)-B.

14. The method of claim 13, wherein said second pharmaceutical composition administered to said subject at least 7 days after said first pharmaceutical composition is administered.

15. The method of claim 1, wherein said ASC express a marker selected from the group consisting of CD73, CD90, CD29 and CD105.

16. The method of claim 1, wherein said ASC do not express a marker selected from the group consisting of CD3, CD4, CD11b, CD14, CD19, and CD34.

17. The method of claim 1, wherein said ASC do not express a marker selected from the group consisting of CD3, CD4, CD34, CD39, and CD106.

18. The method of claim 17, wherein less than 50% of said ASC express CD200.

19. The method of claim 17, wherein more than 50% of said ASC express CD200.

20. The method of claim 17, wherein more than 50% of said ASC express CD141.
